(19) United States
(12) Patent Application Publication
(10) Pub. No.: US 2013/0079245 A1
(43) Pub. Date: Mar. 28, 2013

(54) Biomarkers for Ulcerative Colitis and Crohn's Disease

(71) Applicant: Exagen Diagnostics, Inc., Albuquerque, NM (US)

(72) Inventors: Cole Harris, Houston, TX (US); John Alsobrook, Corrales, NM (US); Lisa Davis, Albuquerque, NM (US)

(73) Assignee: EXAGEN DIAGNOSTICS, INC., Albuquerque, NM (US)

(21) Appl. No.: 13/687,843

(22) Filed: Nov. 28, 2012

Related U.S. Application Data
(63) Continuation of application No. 13/082,712, filed on Apr. 8, 2011.
(60) Provisional application No. 61/322,397, filed on Apr. 9, 2010.

(51) Int. Cl. C12Q 1/68

(52) U.S. Cl. CPC C12Q 1/6876; C12Q 1/6844; C12Q 1/6813
USPC 506/9; 506/16

(57) ABSTRACT

The present invention provides compositions and their use in diagnosing ulcerative colitis, Crohn's Disease, and inflammatory bowel disease.

### RELATED APPLICATIONS

This application is a continuation of U.S. patent application Ser. No. 13/082,712 filed Apr. 8, 2011 which claims priority to U.S. Provisional Patent Application Ser. No. 61/322,397 filed Apr. 9, 2010, incorporated by reference herein in its entirety.

### BACKGROUND

The two major forms of Inflammatory Bowel Disease (IBD) are ulcerative colitis (UC) and Crohn's disease (CD). IBD is a chronic and remitting disease causing inflammation of the intestinal diseases. UC and CD have symptoms and pathologies in common, but they differ in the severity and location of the inflammation along the intestinal tract. Inflammation in UC patients is limited to the mucosal layer, and involves only the rectum and colon, while inflammation in CD patients penetrates the entire wall of the intestine and can occur anywhere along the intestinal tract. A clear diagnosis of the type of IBD is crucial to treatment decisions.

UC typically is characterized by ulcers in the colon and chronic diarrhea mixed with blood, weight loss, blood on rectal examination, and occasionally abdominal pain. UC patients may also present with a variety of other symptoms and extraintestinal manifestations including but not limited to anemia, weight loss, iritis, seronegative arthritis, ankylosing spondylitis, sacroiliitis, erythema nodosum, and pyoderma gangrenosum. Toxic megacolon is a life threatening complication of UC and requires urgent surgical intervention. UC usually requires treatment to go into remission. UC therapy includes anti-inflammatories, immunosuppressants, steroids, and colectomy (partial or total removal of the large bowel, which is considered curative). There is a significantly increased risk of colorectal cancer in UC patients several years after diagnosis, if involvement is beyond the splenic flexure, and a significant risk of primary sclerosing cholangitis, a progressive inflammatory disorder of the bile ducts.

Crohn's disease (CD) is also an IBD feat can affect the colon with symptoms similar to UC. Unlike UC, CD may affect any part of the gastrointestinal tract, and the inflammation penetrates deeper into the layers of the intestinal tact. Patients with CD may have symptoms and intestinal complications including abdominal pain, diarrhea, occult blood, vomiting, weight loss, anemia, fecal incontinence, intestinal obstructions, perianal disease, fistulae, and strictures, and apthous ulcers of the mouth. Extraintestinal complications include skin rashes, arthritis, uveitis, seronegative arthritis, peripheral neuropathy, episcleritis, fatigue, depression, erythema nodosum, pyoderma gangrenosum, growth failure in children, headache, seizures, and lack of concentration. The risk of small intestine malignancy is increased in CD patients. CD is believed to be an autoimmune disease, while it is uncertain whether there is an autoimmune component to UC. There is no known drug or surgical cure for CD; treatment focuses on controlling symptoms and maintaining remission to prevent relapse. Surgery is used for complications of Crohn's (e.g. strictures, fistulae, bleeding), and to remove segments of the intestine with active disease, but there is a high risk of recurrence; thus surgery is not considered curative.

Currently, IBD (such as UC and CD) can only be definitively diagnosed by colonoscopy, a rather invasive procedure; even this invasive procedure is incapable of diagnosing approximately 10% of patients undergoing colonoscopy (Burczynski, J. Mol. Diag. 8 (1): 51 (2006)). It is important to distinguish UC and CD, as disease course and treatment differ, especially with respect to surgical intervention, as noted above.

Thus, there is a need in the art for better and more specific diagnostic tests capable of diagnosing and distinguishing between UC and CD.

### SUMMARY OF THE INVENTION

In a first aspect, the present invention provides biomarkers consisting of between 2 and 35 different nucleic acid probe sets, including:

(a) a first probe set that selectively hybridizes under high stringency conditions to a nucleic add target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), WIPF1 (SEQ ID NO:10), or full complements thereof; and

(b) a second probe set that selectively hybridizes under high stringency conditions to a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8) QARS (SEQ ID NO:9), WIPF1 (SEQ ID NO:10), or full complements thereof;

wherein the first probe set and the second probe set do sot selectively hybridize to the same nucleic acid target.

In a second aspect the present invention provides a biomarker consisting of between 2 and 35 different primer pairs, including:

(a) a first primer pair capable of selectively amplifying a detectable portion of a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4(SEQ ID NO:8), QARS (SEQ ID NO:9), WIPF1 (SEQ ID NO: 10), or full complements thereof; and

(b) a second primer pair capable of selectively amplifying a detectable portion of a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), WIPF1 (SEQ ID NO:10), or full complements thereof,

wherein the first primer pair and the second primer pair do not selectively amplify the same nucleic acid target.

In a third aspect, the present invention provides methods for diagnosing UC and/or CD comprising:

(a) contacting a mRNA-derived nucleic acid sample obtained from a subject suspected of having UC or CD under hybridizing conditions with 2 or more probes sets, wherein at least a first probe set and a second probe set selectively hybridize under high stringency conditions to a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO: 6), HBA2(SEQ ID NO:7), RBM4 (SEQ ID NO:8), QARS (SEQ ID NO:9), WIPF1 (SEQ ID NO: 10), or full complements thereof; wherein the first probe set and the second probe set do not selectively hybridize to the same nucleic acid target;

(b) detecting formation of hybridization complexes between the 2 or more probe sets and nucleic acid targets in the nucleic acid sample, wherein a number of such hybridization complexes provides a measure of gene expression of the nucleic acid targets; and

(c) diagnosing whether the subject is likely to have UC or CD based on the gene expression of the nucleic acid targets.

In one embodiment of the third aspect of the invention, diagnosing whether the subject is likely to have UC or, CD comprises analyzing gene expression of the nucleic acid targets by applying a weight to the number of hybridization complexes formed for each nucleic acid target.

In a fourth aspect, the present invention provides methods for diagnosing UC and/or CD comprising:

(a) contacting a mRNA-derived nucleic acid sample obtained from a subject suspected of having UC or CD under amplifying conditions with 2 or more primer pairs, wherein at least a first primer pair and a second primer pair are capable of selectively amplifying a detectable portion of a nucleic acid target selected from the group consisting of IGH (SEQ ID NO: 11, 12, 13, 14, 15, and/or 16), MMD (SEQ ID NO:2), PDLIM1(SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), WIPF1(SEQ ID NO:10), or full complements thereof; wherein the first, primer pair and the second primer pair do not selectively amplify the same nucleic acid target;

(b) detecting amplification products generated by amplification of nucleic acid targets in the nucleic acid sample by the two or more primer pairs, wherein the amplification products provide a measure of gene expression of the nucleic acid targets; and

(c) diagnosing whether the subject is likely to have UC or CD based on the amplification of the nucleic acid targets.

In one embodiment of the fourth aspect of the invention, diagnosing whether the subject is likely to have UC, CD, or neither based on the amplification of the nucleic acid targets comprises analyzing the amplification products by applying a weight to the number of amplification products formed for each nucleic acid target.

In a preferred embodiment of the third and fourth aspects of the invention, the subject has a diagnosis of IBD, and the method thus comprises distinguishing whether the subject has UC or CD.

In a fifth aspect the present invention provides methods for diagnosing IBD comprising:

(a) contacting a mRNA-derived nucleic acid sample obtained from a subject suspected of having IBD under hybridizing conditions with 2 or more probes sets, wherein at least a first probe set and a second probe set selectively hybridize under high stringency conditions to a nucleic acid target selected from the group consisting of IGH (SEQ ID NO: 11, 12, 13, 14, 15 and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO: 6), HBA2 (SEQ ID NO:7), RBM4 (SEQ ID NO:8), QARS (SEQ ID NO:9), and WIPF1 (SEQ ID NO:10), or full complements thereof; wherein the first probe set and the second probe set do not selectively hybridize to the same nucleic acid target;

(b) detecting formation of hybridization complexes between the 2 or more probe sets and nucleic acid targets in the nucleic acid sample, wherein a number of such hybridization complexes provides a measure of gene expression of the nucleic acid targets; and

(c) diagnosing whether the subject is likely to have IBD based on the gene expression of the nucleic acid targets.

In a sixth aspect, the present invention provides methods method for diagnosing IBD comprising:

(a) contacting a mRNA-derived nucleic acid sample obtained from a subject suspected of having IBD under amplifying conditions with 2 or more primer pairs, wherein at least a first primer pair and a second primer pair are capable of selectively amplifying a detectable portion of a nucleic acid target selected from the group consisting of IGH, (SEQ ID NO: 11, 12, 13, 14, 15, and/or 16), MMD (SEQ ID NO:2), PDLIM1(SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8):, QARS (SEQ ID NO:9) and WIPF1 (SEQ ID NO: 10), or full complements thereof; wherein the first primer pair and the second primer pair do not selectively amplify the same nucleic acid target;

(b) detecting amplification products generated by amplification of nucleic acid targets in the nucleic acid sample by the two or more primer pairs, wherein the amplification products provide a measure of gene expression of the nucleic acid targets; and

(c) diagnosing whether the subject is likely to have IBD based on the amplification of the nucleic acid targets.

In a seventh aspect, the present invention provides methods for diagnosing IBD and providing a differential diagnosis of UC or CD comprising.

(a) contacting a mRNA-derived nucleic acid sample obtained from a subject suspected of having IBD under hybridizing conditions with 2 or more probes sets, wherein at least a first, probe set and a second probe set selectively hybridize under high stringency conditions to a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2(SEQ ID NO:7), RBM4 (SEQ ID NO:8), QARS (SEQ ID NO:9), and WIPF1 (SEQ ID NO:10), or full complements thereof; wherein the first probe set and the second probe set do not selectively hybridize to the same nucleic acid target;

(b) detecting formation of hybridization complexes between the 2 or more probe sets and nucleic acid targets in the nucleic acid sample, wherein a number of such hybridization complexes provides a measure of gene expression of the nucleic acid targets;

(c) diagnosing whether the subject is likely to have IBD based on the gene expression of the nucleic acid targets; and

(d) further diagnosing whether the IBD patient has UC or CD based on the gene expression of the nucleic acid targets.

In an eighth aspect, the present invention provides methods for diagnosing IBD and providing a differential diagnosis of UC or CD comprising:

(a) contacting a mRNA-derived nucleic acid sample obtained from a subject suspected of saving IBD under amplifying conditions with 2 or more primer pairs, wherein at least a first primer pair and a second primer pair are capable of selectively amplifying a detectable portion of a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:5), QARS (SEQ ID NQ:9), and WIPF1 (SEQ ID NO: 10), or full complements thereof; wherein the first primer pair and the second primer pair do not selectively amplify the same nucleic acid target;

(b) detecting amplification products generated by amplification of nucleic acid targets in the nucleic acid sample by the two or more primer pairs, wherein the amplification products provide a measure of gene expression of the nucleic acid targets; and

(c) diagnosing whether the subject is likely to have IBD based on the amplification of the nucleic acid targets; and

(d) further diagnosing whether the IBD patient has UC or CD based on the amplification of the nucleic acid targets.

### DETAILED DESCRIPTION OF THE INVENTION

All references cited are herein incorporated fey reference is their entirety. All embodiments of the invention can be used together in combination unless the context clearly dictates otherwise.

Within this application, unless otherwise stated, the techniques utilized may be found in any of several well-known references such as: Molecular Cloning: A Laboratory Manual (Sambrook, et al, 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Amnion, Austin, Tex.).

In a first aspect, the invention provides biomarkers consisting of between 2 and 35different nucleic acid probe sets, including:

(a) a first probe set mat selectively hybridizes under high stringency conditions to a nucleic acid target selected from the group consisting of IGH (SEQ ID NO: 11, 12, 13, 14, 15, and/or 16) MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), WIPF1 (SEQ ID NO:10), or full complements thereof; and

(b) a second probe set feat selectively hybridizes under high stringency conditions to a nucleic acid target selected from the group consisting of IGH (SEQ ID NO: 11, 12, 13, 14, 15, and/or 16) (SEQ ID NO:1), MMD (SEQ ID NO:2), PDLIM1(SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), WIPF1 (SEQ ID NO:10), or full complements thereof, wherein the first probe set and the second probe set do not selectively hybridize to the same nucleic acid target.

The recited nucleic acid targets are human nucleic acids recited by SEQ ID NO and gene name; as will be understood by those of skill in the art, such human nucleic acid sequences also include the mRNA counterpart to the sequences disclosed herein. For ease of reference, the nucleic acids will be referred to by gene name throughout the rest of the specification; it will be understood that as used herein the gene name means the recited SEQ. ID. NOS. for each gene listed in Table 1, complements thereof, and RNA counterparts thereof.

In one non-limiting example, the first probe set selectively hybridizes under high stringency conditions to CD4, and thus selectively hybridizes under high stringency conditions to the nucleic acid of SEQ ID NO:5 (NCBI Reference Sequence number NM_{—}000616.3) a mRNA version thereof, or complements thereof, and the second probe set selectively hybridizes under high stringency conditions to MMD (NCBI Reference Sequence number NM_{—}012329.2, thus selectively hybridizing under high stringency conditions to the nucleic acid of SEQ ID NO: 2, a mRNA version thereof, or complements thereof. Further embodiments will be readily apparent to those of skill is the art based on the teachings herein and Table 1 below.

In this and all other aspects and embodiments, recitation of “IGH (SEQ ID NO: 11, 12, 13, 14, 15, and/or 16)” means that “IGH” is one “nucleic acid target” of the recited set of nucleic acid targets (in this case, a set of 10 nucleic acid targets), represented by 6 different nucleic acid sequences (SEQ ID NOS: 11 (IGHG3), 12 (IGHG1), 13 (IGHM), 14 (IGH@), 15 (IGHV4-31), and/or 16 (IGHG4)). Thus, a first probe set that selectively hybridizes under high stringency conditions to IGH may include probes for SEQ ID NO:11 only, 11 and 12 only; 12 only; each of 11, 12, 13, 14, 15, and 16; or any other combination thereof).

TABLE 1
Nucleic acid sequences

| HGNC | | | GenBank | | |
|---|---|---|---|---|---|
| gene | Chromosome | NCBI Reference | Accession | | |
| symbol | Location | Sequence | Number | HGNC Gene Name | Alias |
| IGHG3 | 14q32.33 | NG_001019.5 | M87789.1 | immunoglobulin | anti-hepatitis A |
| | | (SEQ ID NO: 1A) | | heavy constant | IgG variable |
| | | | | gamma 3 (G3m | region, constant |
| | | | | marker) | region, |
| IGHG1 | 14q32.33 | NG_001019.5 | BC067091.1 | immunoglobulin | |
| | | (SEQ ID NO: 1B) | | heavy constant | |
| | | | | gamma 1 (G1m | |
| | | | | marker) | |
| IGHM | 14q32.33 | NG_001019.5 | BC016381.1 | immunoglobulin | |
| | | (SEQ ID NO: 1C) | | heavy constant mu | |
| IGH@ | 14q32.33 | NG_001019.5 | BC073766.1 | immunoglobulin | |
| | | (SEQ ID NO: 1D) | | heavy locus | |
| IGHV4- | 14q32.33 | NG_001019.5 | BC073773.1 | immunoglobulin | |
| 31 | | (SEQ ID NO: 1E) | | heavy variable 4-31 | |
| IGHG4 | 14q32.33 | NG_001019.5 | BC025985.1 | immunoglobulin | |
| | | (SEQ ID NO: 1F) | | heavy constant | |
| | | | | gamma 4 (G4m | |
| | | | | marker) | |
| MMD | 17q | NM_012329.2 | | monocyte to | MMA, PAQR11 |
| | | (SEQ ID NO: 2) | | macrophage | |
| | | | | differentiation- | |
| | | | | associated | |
| PDLIM1 | 10q23.1 | NM_020992.2 | | PDZ and LIM | CLP-36, |
| | | (SEQ ID NO: 3) | | domain 1 | hCLIM1, |
| | | | | | CLP36 |
| PDIA6 | 2p25.1 | NM_005742.2 | | protein disulfide | P5, ERp5 |
| | | (SEQ ID NO: 4) | | isomerase family A, | |
| | | | | member 6 | |
| CD4 | 12pter-p12 | NM_000616.3 | | CD4 molecule | |
| | | (SEQ ID NO: 5) | | | |
| DNAJA1 | 9p13.3 | NM_001539.2 | | DnaJ (Hsp40) | HSPF4, hdj-2, |
| | | (SEQ ID NO: 6) | | homolog, subfamily | dj-2 |
| | | | | A, member 1 | |
| HBA2 | 16p13.3 | NM_000517.4 | | hemoglobin, alpha 2 | |
| | | (SEQ ID NO: 7) | | | |
| RBM4 | 11q13 | NM_002896.2 | | RNA binding motif | LARK, |
| | | (SEQ ID NO: 8) | | protein 4 | RBM4A, |
| | | | | | ZCRB3A, |
| | | | | | ZCCHC21 |
| QARS | 3p21.31 | NM_005051.1 | | glutaminyl-tRNA | |
| | | (SEQ ID NO: 9) | | synthetase | |
| WIPF1 | 2q31.2 | NM_001077269.1 | | WAS/WASL | WIP |
| | | (SEQ ID NO: 10) | | interacting protein | |
| | | | | family, member 1 | |

As is described in more detail below, the inventors have discovered that the biomarkers of the invention can be used, for example, as probes for diagnosing and distinguishing UC and CD, which is critical for making treatment decisions for such subjects. The biomarkers can be used, for example, to determine the expression levels in tissue of mRNA for the recited genes. The biomarkers offers first aspect of the invention are especially preferred for use in RNA expression analysis from the genes hi a tissue of interest, such as blood samples (for example, peripheral blood mononuclear cells (PBMCs)s RBC-depleted whole blood, or lysed whole blood).

As used herein with respect to all aspects and embodiments of the invention, a “probe set” is one or more isolated polynucleotides that each selectively hybridize under high stringency conditions to the same target nucleic acid target (for example, a single specific mRNA). Thus, a single “probe set” may comprise any number of different isolated polynucleotides that selectively hybridize under high stringency conditions to the same nucleic acid target, such as an mRNA expression product. For example, a probe set that selectively hybridizes to a CD4 mRNA may consist of a single polynucleotide of 100nucleotides that selectively hybridizes under high stringency conditions to CD4 mRNA, may consist of two separate polynucleotides 100 nucleotides in length that each selectively hybridise under high stringency conditions to CD4 mRNA, or may consist of twenty separate polynucleotides 25 nucleotides in length that each selectively hybridize under high stringency conditions to CD4 mRNA (such as, for example, fragmenting a larger probe into many individual shorter polynucleotides). Those of skill in the art will understand that many such permutations are possible. For purposes of the present invention, “IGH” is considered a single nucleic acid target, such that a single probe set may include isolated polynucleotides that selectively hybridize under high stringency conditions to 1, 2, 3, 4, 5, or all 6 of SEQ ID NOS: 11, 12, 13, 14, 15, and 16.

The biomarkers of the invention consist of between 2 and 35 probe sets. In various embodiments, the biomarker can include 3, 4, 5, 6, 7, 8, 9, or 10 probe sets that selectively hybridise under high stringency conditions to a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof, wherein each of the 3-10 different probe sets selectively hybridize under high stringency conditions to a different nucleic acid target. Thus, as will be clear to those of skill in the art, the biomarkers may include further probe sets that, for example, (a) are additional probe sets that also selectively hybridize under high stringency conditions to the recited human nucleic acid target; or (b) do not selectively hybridize under high stringency conditions to any of the recited human nucleic acid targets. Such further probe sets of type (b) may include those consisting of polynucleotides that selectively hybridize to other nucleic acids of interest, such as those targeting internal reference genes used for normalization, and may further include, for example, probe sets consisting of control sequences, such as competitor nucleic acids. Further, one skilled in the art that the probe sets may be hybridized control materials of known concentrations to define a standard curve for quantitating the expression levels of test samples.

In various embodiments of this first aspect, the biomarker consists of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 probe sets, hi various further embodiments, at least 10%, 15%, 20%, 25%, 30%, 35%, 40%, 45%, 50%, 55%, 60%, 65%, 70%, 75%, 80%, 85%, 90%, 95%, or more of the different probe sets selectively hybridize under high stringency conditions to a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof.

As will be apparent to those of skill in the art, as the percentage of probe sets that selectively hybridize under high stringency conditions to a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO: 10), or full complements thereof increases, the maximum number of probe sets in the biomarker will decrease accordingly. Thus, for example, where at least 50% of the probe sets selectively hybridize under high stringency conditions to a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2(SEQ ID NO:7), RBM4 (SEQ ID NO:8), QARS (SEQ ID NO:9), and WIPF1 (SEQ ID NO:10), or their complements, the biomarker will consist of between 2 and 20 probe sets. Those of skill in the art will recognize the various other permutations encompassed by the compositions according to the various embodiments of this aspect of the invention.

As used herein with respect to each aspect and embodiment of the invention, the term “selectively hybridizes” means that the isolated polynucleotides are fully complementary to at least a portion of their nucleic acid target so as to form a detectable hybridization complex under the recited hybridization conditions, where the resulting hybridization complex is distinguishable from any hybridization that might occur with other nucleic acids. The specific hybridization conditions used will depend on the length of the polynucleotide probes employed, their GC content, as well as various other factors as is well known to those of skill in the art. (See, for example, Tijssen (1993) Laboratory Techniques in Biochemistry and Molecular Biology—Hybridization with Nucleic Acid Probes part I, chapter 2, “Overview of principles of hybridization and the strategy of nucleic acid probe assays,” Elsevier, N.Y. (“Tijssen”)). As used herein, “stringent hybridization conditions” are selected to be no more than 5° C. lower than the thermal melting point (Tm) for the specific polynucleotide at a defined ionic strength and pH. The Tm is the temperature (under defined ionic strength and pH) at which 50% of the target sequence hybridizes to a perfectly matched probe. High stringency conditions are selected to be equal to the Tm for a particular polynucleotide probe. An example of stringent conditions are those that permit selective hybridization of the isolated polynucleotides to the genomic or other target nucleic acid to form hybridization complexes in 0.2×SSC at 65° C. for a desired period of time, and wash conditions of 0.2×SSC at 65° C. for 15 minutes. It is understood that these conditions may be duplicated using a variety of buffers and temperatures. SSC (see, e.g., Sambrook, Fritsch, and Maniatis, in: Molecular Cloning, A Laboratory Manual, Cold Spring Harbor Laboratory Press, 1989) is well known to those of skill in the art, as are other suitable hybridization buffers.

The polynucleotides in the probe sets can be of any length that permits selective hybridization under high stringency conditions to the nucleic acid target of interest, or full complements thereof. In various preferred embodiments of this aspect of the invention and related aspects and embodiments disclosed below, the isolated polynucleotides are at least 10, 15, 20, 25, 30, 35, 40, 45, 50, 55, 60, 65, 70, 75, 80, 85, 90, 95, 100, 150, 200, 250, 300, 350, 400, 459, 500, 550, 600, 650, 700, 759, 800, 850, 900, 950, 1000, 1100, 1200, 1300, 1400, 1500, 1600, 1700, 1800, 1900, 2000, or more nucleotides in length of one of the recited SEQ ID NOS for the nucleic acid target of interest, full complements thereof, or corresponding RNA sequences.

The term “polynucleotide” as used herein refers to DNA or RNA, preferably DNA, in either single- or double-stranded form. In a preferred embodiment, the polynucleotides are single stranded nucleic acids that are “anti-sense” to the recited nucleic acid (or its corresponding RNA sequence). The term “polynucleotide” encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs), methylphosphonate linkages or alternating methylphosphonate and phosphodiester linkages (Strauss-Soukup (1997) Biochemistry 36:8692-8698), and benzylphosphonate linkages, as discussed in U.S. Pat. No. 6,664,057; see also Oligonocleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press).

An “isolated” polynucleotide as used herein for all of the aspects and embodiments of the invention is one which is free of sequences which naturally Sank the polynucleotide in the genomic DNA of the organism from which the nucleic acid is derived, and preferably free from linker sequences found in nucleic acid libraries, such as cDNA libraries. Moreover, an “isolated” polynucleotide is substantially free of other cellular material, gel materials, and culture medium when produced by recombinant techniques, or substantially free of chemical precursors or other chemicals when chemically synthesized. The polynucleotides of the invention may be isolated from a variety of sources, such as by PCR amplification from genomic DNA, mRNA, or cDNA libraries derived from mRNA, using standard techniques; or they may be synthesized in vitro, by methods well known to those of skill is the art, as discussed in U.S. Pat. No. 6,664,057 and references disclosed therein. Synthetic polynucleotides can be prepared by a variety of solution or solid phase methods. Detailed descriptions of the procedures for solid phase synthesis of polynucleotide by phosphite-triester, phosphotriester, and H-phosphonate chemistries are widely available. (See, for example, U.S. Pat. No. 6,664,057 and references disclosed therein). Methods to purify polynucleotides include native acrylamide gel electrophoresis, and anion-exchange HPLC, as described in Pearson (1983) J. Chrom. 255:137-149, the sequence of the synthetic polynucleotides can be verified using standard methods.

In one preferred embodiment, the polynucleotides are double or single stranded nucleic acids that include a strand that is “anti-sense” to all or a portion of the SEQ ID NOS shown above for each gene of interest or its corresponding RNA sequence (ie: it is fully complementary to the recited SEQ ID NOs). In one non-limiting example, the first probe set selectively hybridizes under high stringency conditions to IGHG3, and is fully complementary to all or a portion of the nucleic acid of SEQ ID NO:1, a full complement thereof, or a mRNA version thereof, and the second probe set selectively hybridizes under high stringency conditions to MMD and is fully complementary to the nucleic acid of SEQ ID NO: 2, a full complement thereof, or a mRNA version thereof.

In one preferred embodiment of this first aspect of the invention, the biomarker includes a first probe set that selectively hybridizes under high stringency conditions to CD4 (SEQ ID NO:5), or a full complement thereof, a second probe set that selectively hybridizes under high stringency conditions to DNAJA1 (SEQ ID NO:6), or a full complement thereof, and a third probe set that selectively hybridizes under high stringency conditions to MMD (SEQ ID NO: 2), or a full complement thereof. As disclosed in more detail below, the inventors have discovered that such biomarkers are particularly useful as probes to distinguish between UC and CD patients.

In a second preferred embodiment of this first aspect of the invention, the biomarker includes a first probe set that selectively hybridizes under high stringency conditions to MMD (SEQ ID NO:2), or a full complement thereof, a second probe set that selectively hybridizes under high stringency conditions to PDIA6 (SEQ ID NO:4), or a full complement thereof, a third probe set that selectively hybridizes under high stringency conditions to QARS (SEQ ID NO:9), or a full complement thereof, and a fourth probe set that selectively hybridizes under high stringency conditions to WIPF1 (SEQ ID NO:10), or a full complement thereof. As disclosed in more detail below, the inventors have discovered that such biomarkers are particularly useful as probes to distinguish between UC and CD patients.

In a third preferred embodiment of this first aspect of the invention, the biomarker includes a first probe set selectively hybridizes under high stringency conditions to MMD (SEQ ID NO:2), or a full complement thereof, a second probe set selectively hybridizes under high stringency conditions to PDIA6 (SEQ ID NO:4), or a full complement thereof, a third probe set selectively hybridizes under high stringency conditions to QARS (SEQ ID NO:9), or a full complement thereof, and a fourth probe set selectively hybridizes under high stringency conditions to one or more of SEQ ID NO:11, SEQ ID NO:12, SEQ ID NO:13, SEQ ID NO:14, SEQ ID NO:15, and/or SEQ ID NO:16 (IGH), or full complements thereof. As disclosed in more detail below, the inventors have discovered that such biomarkers are particularly useful as probes to distinguish between UC and CD patients, and also have been found particularly useful for distinguishing normal subjects from those having inflammatory bowel disease. In one preferred embodiment, the fourth probe set selectively hybridizes under high stringency conditions to one or more of SEQ ID NO:11 (IGHG3); in another preferred embodiment, the fourth probe set selectively hybridizes to each of SEQ ID NO:11, SEQ ID NO:12, SEQ ID NO:13, SEQ ID NO:14, SEQ ID NO:15, and SEQ ID NO:16 (IGH):, as SEQ ID NOS: 11, 12, 13, 14, 15, and 16 share adequate sequence identify to enable those of skill in the art to design one or more probes that hybridize under high stringency conditions to each.

In a fourth preferred embodiment of this first aspect of the invention, the biomarker includes a first probe set that selectively hybridizes under high stringency conditions to PDLIM1 (SEQ ID NO:3), or a full complement thereof, a second probe set that selectively hybridizes under high stringency conditions to PDIA6 (SEQ ID NO:4), or a full complement thereof, a third probe set that selectively hybridizes under high stringency conditions to RBM4 (SEQ ID NO:8), or a full complement thereof, a fourth probe set that selectively hybridizes under high stringency conditions to QARS (SEQ ID NO:9), or a full complement thereof, and a fifth probe set that selectively hybridizes under high stringency conditions to WIPF1 (SEQ ID NO: 10), or a full complement thereof. As disclosed in more detail below, the inventors have discovered that such biomarkers are particularly useful as probes to distinguish between normal subjects from those having inflammatory bowel disease.

In a fifth preferred embodiment of this first aspect of the invention, the biomarker includes a first probe set that selectively hybridizes under high stringency conditions to CD4 (SEQ ID NO: 5), or a full complement thereof, a second probe set that selectively hybridizes under high stringency conditions to DNAJA1 (SEQ ID NO:6), or a full complement thereof, a third probe set that selectively hybridizes under high stringency conditions to MMD (SEQ ID NO:2), or a fall complement thereof, a fourth probe set that selectively hybridizes under high stringency conditions to RBM4 (SEQ ID NO:8), or a full complement thereof, and a fifth probe set that selectively hybridizes under high stringency conditions to WIPF1 (SEQ ID NO:10), or a full complement thereof. As disclosed in more detail below, the inventors have discovered that such biomarkers are particularly useful as probes to distinguish between UC and CD patients.

In a sixth preferred embodiment of this first aspect of the invention, the biomarker includes a first probe set that selectively hybridizes under high stringency conditions to CD4 (SEQ ID NO:5), or a full complement thereof, a second probe set that selectively hybridizes under high stringency conditions to PDLIM1 (SEQ ID NO:3), or a full complement thereof, and a third probe set that selectively hybridizes under high stringency conditions to RBM4 (SEQ ID NO:8), or a full complement thereof. As disclosed is more detail below, the inventors have discovered that such biomarkers are particularly useful as probes for diagnosing IBD.

In a second aspect, the present invention provides biomarkers comprising or consisting of between 2 and 35 different nucleic acid primer pairs, wherein

(a) a first primer pair capable of selectively amplifying a detectable portion of a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO: 10), or full complements thereof; and

(b) a second primer pair capable of selectively amplifying a detectable portion of a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof;

wherein the first primer pair and the second primer pair do not selectively amplify the same nucleic acid target.

As is described in more detail below, the inventors have discovered that the biomarkers of the invention can be used, for example, as primers for amplification assays for diagnosing/distinguishing UC and CD. The biomarkers can be used, for example, to determine the expression levels in tissue of mRNA for the recited genes. The biomarkers of this second aspect of the invention are especially preferred for use in RNA expression analysis from the genes in a tissue of interest, such as blood, samples (PBMCs, RBC-depleted whole blood, or lysed whole blood).

The nucleic acid targets have been described in detail above, as have polynucleotides in general. As used herein, “selectively amplifying” means that the primer pairs are complementary to their targets and can be used to amplify a detectable portion of the nucleic acid target that is distinguishable from amplification products due to non-specific amplification. In a preferred embodiment, the primers are fully complementary to their target.

For purposes of the present invention, “IGH” is considered a single nucleic acid target, such that a primer pair may include isolated polynucleotides that selectively amplify a detectable portion of 1, 2, 3, 4, 5, or all 6 of SEQ ID NOS: 11, 12, 13, 14, 15, and 16.

As is well known in the art, polynucleotide primers can be used is various assays (PCR, RT-PCR, RTQ-PCR, spPCR, qPCR, qRT-PCR, and allele-specific PCR, etc.) to amplify portions of a target to which me primers are complementary. Thus, a primer pair would include both a “forward” and a “reverse” primer, one complementary to the sense strand (ie: the stand shown in the sequences provided herein) and one complementary to an “anti-sense” strand (ie: a strand complementary to the strand shown in the sequences provided herein), and designed to hybridize to the target so as to be capable of generating a detectable amplification product from the target of interest when subjected to amplification conditions. The sequences of each of the target nucleic acids are provided herein, and thus, based on the teachings of the present specification, those of skill in the art can design appropriate primer pairs complementary to the target of interest (or complements thereof). In various preferred embodiments, each member of the primer pair is a single stranded DNA polynucleotide at least 15, 16, 17, 18, 19, 20, 21, 22, 23, 24, 25, 26, 27, 28, 29, 30, or more nucleotides in length that are fully complementary to the nucleic acid target. In various further embodiments, the detectable portion of the target nucleic acid that is amplified is at least 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or more nucleotides in length.

In various embodiments, me biomarker can comprise or consist of 3, 4, 5, 6, 7, 8, 9, or 10 primer pairs that selectively amplify a detectable portion of a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof, wherein none of the 3-10 primer pairs selectively amplify the same nucleic acid target. In a preferred embodiment, the primers are fully complementary to their target. As will be clear to those of skill in the art, the biomarkers may include further primer pairs that do not selectively amplify any of the recited human nucleic acid targets. Such further primer pairs may include those consisting of polynucleotides that selectively amplify other nucleic acids of interest, such as those targeting internal reference genes used for normalization, and may further be used to amplify control materials of known concentrations to define a standard curve for quantitating the expression levels of test samples.

In various embodiments of this second aspect, the biomarker consists of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 primer pairs. In various further embodiments, at least 10%, 15%, 20%, 25%, 30%, 35%, 40%, 45%, 50%, 55%, 60%, 65%, 70%, 75%, 80%, 85%, 90%, 95%, or more of the different punier pairs selectively amplify a detectable portion of a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ED NO:6), HBA2 (SEQ ID NO:7), RBM4 (SEQ ID NO:8), QARS (SEQ ID NO:9), and WIPF1 (SEQ ID NO: 10), or full complements thereof.

In one preferred embodiment a biomarker according to this second aspect of the invention comprises or consists of a first primer pair that selectively amplifies a detectable portion of CD4 (SEQ ID NO: 5), or a full complement thereof; a second primer pair that selectively amplifies a detectable portion of DNAJA1 (SEQ ID NO:6), or a full complement thereof, and a third primer pair that selectively amplifies a detectable portion of MMD (SEQ ID NO:2) or a full complement thereof. As disclosed in more detail below, the inventors have discovered that such biomarkers can be used as probes to distinguish between UC and CD patients.

In a second preferred embodiment a biomarker according to this second aspect of the invention comprises or consists of a first primer pair that selectively amplifies a detectable portion of MMD (SEQ ID NO:2) or a full complement thereof; a second primer pair that selectively amplifies a detectable portion of PDIA6 (SEQ ID NO:4) or a full complement thereof; a third primer pair that selectively amplifies a detectable portion of QARS (SEQ ID NO:9) or a full complement thereof; and a fourth primer pair that selectively amplifies a detectable portion of WIPF1 (SEQ ID NO:10) or a full complement thereof. As disclosed in more detail, below, the inventors have discovered that such biomarkers are particularly useful as probes to distinguish between UC and CD patients.

In a third preferred embodiment, a biomarker according to this second aspect of the invention comprises or consists of a first primer pair that selectively amplifies a detectable portion of MMD (SEQ ID NO:2) or a full complement thereof; a second primer pair that selectively amplifies a detectable portion of PDIA6 (SEQ ID NO:4) or a full complement thereof; a third primer pair that selectively amplifies a detectable portion of PDIA6 (SEQ ID NO:4) or a full complement thereof; and a fourth primer pair that selectively amplifies a detectable portion of one or more of SEQ ID NO:11, SEQ ID NO:12, SEQ ID NO:13, SEQ ID NO:14, SEQ ID NO:15, and/or SEQ ID NO:16 (IGH), or full complement thereof. As disclosed in more detail below, the inventors have discovered that such biomarkers are particularly useful as probes to distinguish between UC and CD patients, and also have been found particularly useful for distinguishing normal subjects from those having IBD. In one preferred embodiment the fourth primer pair selectively amplifies SEQ ID NO:11 (IGHG3). In another preferred embodiment, the fourth primer pair selectively amplifies each of SEQ ID NO:11, SEQ ID NO:12, SEQ 13 NO:14, SEQ ID NO:15, and SEQ ID NO:16.

In a fourth preferred embodiment, a biomarker according to this second aspect of the invention comprises or consists of a first primer pair that selectively amplifies a detectable portion of PDLIM1 (SEQ ID NO:3) or a full complement thereof, a second punier pair that selectively amplifies a detectable portion of PDIA6 (SEQ ID NO:4) or a full complement thereof; a third primer pair that selectively amplifies a detectable portion of RBM4 (SEQ ID NO:8) or a full complement thereof; a fourth primer pair that selectively amplifies a detectable portion of QARS (SEQ ID NO:9) or a full complement thereof; and a fifth primer pair that selectively amplifies a detectable portion of WIPF1 (SEQ ID NO:10) or a full complement thereof. As disclosed in more detail below, the inventors have discovered that such biomarkers are particularly useful as probes to distinguish between normal subjects from those having IBD.

In a fifth preferred embodiment of this first aspect of the invention, a biomarker according to this second aspect of the invention comprises or consists of a first primer pair that selectively amplifies a detectable portion of CD4 (SEQ ID NO:5), or a full complement thereof, a second primer pair that selectively amplifies a detectable portion of DNAJA1 (SEQ ID NO:6), or a full complement thereof, a third primer pair that selectively amplifies a detectable portion of MMD (SEQ ID NO:2), or a full complement thereof, a fourth primer pair that selectively amplifies a detectable portion of RBM4 (SEQ ID NO:8), or a full complement thereof, and a fifth primer pair that selectively amplifies a detectable portion of WIPF1 (SEQ ID NO:10), or a full complement thereof. As disclosed in more detail below, the investors have discovered that such biomarkers are particularly useful as probes to distinguish between UC and CD patients.

In a sixth preferred embodiment of this first aspect of the invention, a biomarker according to this second aspect of the invention comprises or consists of a first primer pair that selectively amplifies a detectable portion of CD4 (SEQ ID NO:5), or a full complement thereof, a second primer pair that selectively amplifies a detectable portion of PDLIM1 (SEQ ID NO:3), or a full complement thereof, and a third primer pair that selectively amplifies a detectable portion of RBM4 (SEQ ID NO:8), or a full complement thereof. As disclosed in more detail below, the inventors have discovered that such biomarkers are particularly useful as probes for diagnosing IBD.

The biomarkers of the first and second aspects of the invention can be stored frozen, is lyophilized form, or as a solution containing the different probe sets. Such a solution can be made as such, or the composition can be prepared at the time of hybridizing the polynucleotides to target, as discussed below. Alternatively, the compositions can be placed on a solid support, such as in a microarray or microplate format.

In all of the above aspects and embodiments, the polynucleotides can be labeled with a detectable label. In a preferred embodiment, the detectable labels for polynucleotides in different probe sets are distinguishable from each other to, for example, facilitate differential determination of their signals when conducting hybridization reactions using multiple probe sets. Methods for detecting the label include, but are not limited to spectroscopic, photochemical, biochemical, immunochemical, physical or chemical techniques. For example, useful detectable labels include but are not limited to radioactive labels such as ³²P, ³H, and ¹⁴C; fluorescent dyes such as fluorescein isothiocyasate (FITC), rhodamine, lanthanide phosphors, and Texas red, ALEXIS™ (Abbott Labs), CY™ dyes (Amersham); electron-dense reagents such as gold; enzymes such as horseradish peroxidase, beta-galactosidase, luciferase, and alkaline phosphatase; colorimetric labels such as colloidal gold; magnetic labels such as those sold under the mark DYNABEADS™; biotin; digoxigenin; or haptens and proteins for which antisera or monoclonal antibodies are available. The label can be directly incorporated into the polynucleotide, or it can be attached to a probe or antibody which hybridizes or binds to the polynucleotide. The labels may be coupled to the probes by any suitable means known to those of skill in the art. In various embodiments, the polynucleotides are labeled using nick translation, PCR, or random primer extension (see, e.g., Sambrook et al. supra).

In a third aspect, the present invention provides methods for diagnosing UC or CD comprising:

(a) contacting a mRNA-derived nucleic acid sample obtained from a subject suspected of having UC or CD under hybridizing conditions with 2 or more probes sets, wherein at least a first probe set and a second probe set selectively hybridize under high stringency conditions to a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO: 8), QARS (SEQ ID NO:9), and WIPF1 (SEQ ID NO:10), or fell complements thereof; wherein the first probe set and the second probe set do not selectively hybridize to the same nucleic acid target;

(b) detecting formation of hybridization complexes between the 2 or more probe sets and nucleic acid targets in the nucleic acid sample, wherein a number of such hybridization complexes provides a measure of gene expression of the nucleic acid targets; and

(c) diagnosing whether the subject is likely to have UC or, CD based on the gene expression of the nucleic acid targets.

The inventors have discovered that the methods of the invention can be used, for example, in diagnosing and distinguishing UC and CD. The specific genes, probe sets, hybridizing conditions, probe types, polynucleotides, etc. are as defined above for the first and/or second aspects of the invention.

The subject is any human subject that may be suffering from UC or CD. As discussed above, UC typically is characterized by ulcers in the colon and chronic diarrhea mixed with blood, weight loss, blood on rectal examination, and occasionally abdominal pain. UC patients may also present with a variety of other symptoms, including but not limited to iritis, seronegative arthritis, ankylosing spondylitis, sacroiliitis, erythema nodosum, and pyoderma gangrenosum. CD is usually characterized by abdominal pain, diarrhea (which may be bloody), vomiting, weight loss, skin rashes, arthritis, uveitis, seronegative arthritis, peripheral neuropathy, headache, seizures, episcleritis, fatigue, depression, erythema nodosum, pyoderma gangrenosum, perianal discomfort, fecal incontinence, apthous ulcers of the mouth, growth failure in children, and lack of concentration. Thus, subjects with one or more of these symptoms would be candidate subjects for the methods of the invention.

As used herein, “diagnosing” includes both diagnosing whether a subject has UC or CD, as well as diagnosing whether a subject with an established diagnosis of IBD has UC or CD. In a preferred embodiment of the third aspect of the invention, the subject has a diagnosis of IBD, and the diagnosis thus comprises distinguishing whether the subject has UC or CD.

As used herein, a “mRNA-derived nucleic acid sample” is a sample containing mRNA from the subject, or a cDNA (single or double stranded) generated from the mRNA obtained from the subject. The sample can be from any suitable tissue source, including but not limited to Mood samples, such as PBMCs, RBC-depleted whole blood, or lysed whole blood.

In one embodiment, the mRNA sample is a human mRNA sample. It will be understood by those of skill in the art that the RNA sample does not require isolation of an individual or several individual species of RNA molecules, as a complex sample mixture containing RNA to be tested can be used, such as a cell or tissue sample analyzed by in site hybridization.

In a former embodiment, the probe sets comprise single stranded anti-sense polynucleotides of the nucleic acid compositions of the invention. For example, in mRNA fluorescence in situ hybridization (FISH) (i.e. FISH to detect messenger RNA), only an anti-sense probe strand hybridizes to the single stranded mRNA in the RNA sample, and in that embodiment, the “sense” strand oligonucleotide can be used as a negative control.

Alternatively, the probe sets may comprise DNA probes. In either of these embodiments (anti-sense probes or cDNA probes), it is preferable to use controls or processes that direct hybridization to either cytoplasmic mRNA or nuclear DNA. In the absence of directed hybridization, it is preferable to distinguish between hybridization to cytoplasmic RNA and hybridization to nuclear DNA.

Any method for evaluating the presence or absence of hybridization products in the sample can be used, such as by Northern blotting methods, in situ hybridization (for example, on blood smears), polymerase chain reaction (PCR) analysis, qPCR (quantitative PCR), RT-PCR (Real Time PCR), qRT-PCR (quantitative RT-PCR) or array based methods.

In one embodiment, detection is performed by in situ hybridization (“ISH”). In situ hybridization assays are well known to those of skill in the art. Generally, in situ hybridization comprises the following major steps (see, for example, U.S. Pat. No. 6,664,057): (1) fixation of sample or nucleic acid sample to be analyzed; (2) pre-hybridization treatment of the sample or nucleic acid sample to increase accessibility of the nucleic acid sample (within the sample in those embodiments) and to reduce nonspecific binding; (3) hybridization of the probe sets to the nucleic acid sample; (4) post-hybridization washes to remove polynucleotides not bound in the hybridization; and (5) detection of the hybridized nucleic acid fragments. The reagent used in each of these steps and their conditions for use varies depending on the particular application. In a particularly preferred embodiment, ISH is conducted according to methods disclosed in U.S. Pat. Nos. 5,750,340 and/or 6,022,689, incorporated by reference herein in their entirety.

In a typical in situ hybridization assay, cells are fixed to a solid support typically a glass slide. The cells are typically denatured with heat or alkali and then contacted with a hybridization solution to permit annealing of labeled probes specific to the nucleic acid sequence encoding the protein. The polynucleotides of the invention are typically labeled, as discussed above. In some applications it is necessary to block the hybridization capacity of repetitive sequences. In this case, human genomic DNA or Cot-1 DNA is used to block non-specific hybridization.

When performing an in situ hybridization to cells fixed on a solid support, typically a glass slide, it is preferable to distinguish between hybridization to cytoplasmic RNA and hybridization to nuclear DNA. There are two major criteria for making ins distinction: (1) copy number differences between the types of targets (hundreds to thousands of copies of RNA vs. two copies of DNA) which will normally create significant differences in signal intensities and (2) clear morphological distinction between the cytoplasm (where hybridization to RNA targets would occur) and the nucleus will make signal location unambiguous. Thus, when using double stranded DNA probes, it is preferred that the method further comprises distinguishing the cytoplasm and nucleus in cells being analyzed, within the bodily fluid sample. Such distinguishing can be accomplished by any means known in the art, such as by using a nuclear stain such as Hoeschst 33342 or DAPI, which delineate the nuclear DNA in the cells being analyzed. In this embodiment, it is preferred that the nuclear stain is distinguishable from the detectable probe. It is further preferred that the nuclear membrane be maintained, i.e. that ail the Hoeschst or DAPI stain be maintained in the visible structure of the nucleus.

In a further embodiment, an array-based format can be used in which the probe sets can be arrayed on a surface and the RNA sample is hybridized to the polynucleotides on the surface. In this type of format, large numbers of different hybridization reactions can be run essentially “in parallel”. This embodiment is particularly useful when there are many genes whose expressions in one specimen are to be measured, or when isolated nucleic acid from the specimen, but not the intact specimen, is available. This provides rapid, essentially simultaneous, evaluation of a large number of gene expression assays. Methods of performing hybridization reactions in array based formats are also described in, for example, Pastinen (1997) Genome Res. 7:606-614; (1997) Jackson (1996) Nature Biotechnology 14:1685; Chee (1995) Science 274:610; WO 96/17958. Methods for immobilizing the polynucleotides on the surface and derivatizing the surface are known in the art; see, for example, U.S. Pat. No. 6,664,057.

In each of the above aspects and embodiments, detection of hybridization is typically accomplished through the use of a detectable label on the polynucleotides in the probe sets, such as those described above; in some alternatives, the label can be on the target nucleic acids. The label can be directly incorporated into the polynucleotide, or it can be attached to a probe or antibody which hybridizes or binds to the polynucleotide. The labels may be coupled to the probes in a variety of means knows to those of skill is the art, as described above. The label can be detected by any suitable technique, including but not limited to spectroscopic, fluorescent, photochemical, biochemical, immunochemical, physical, or chemical techniques, as discussed above.

The methods may comprise comparing gene expression of the nucleic acid targets to a control. Any suitable control known in the art can be used in the methods of the invention. For example, the expression level of a gene known to be expressed at a relatively constant level in UC, CD, and normal patients can be used for comparison. Another embodiment is the use of a standard concentration curve that gives absolute copy numbers of the mRNA of the gene being assayed; this might obviate the need for a normalization control because the expression levels would be given in terms of standard concentration units. Those of skill in the art will recognize that many such controls can be used in the methods of the invention.

The methods comprise either (a) diagnosing whether the subject is likely to have UC or CD; or (b) distinguishing whether a subject with an established diagnosis of IBD has UC or CD, based on the gene expression of the nucleic acid target. As used herein, “likely to have” means a statistically significant likelihood that the diagnosis is correct. In various embodiments, the method results in an accurate diagnosis in at least 70% of cases; more preferably of at least 75%, 80%, 85%, 90%, or more of the cases.

The methods of the present invention may apply weights, derived by various means in the art, to the number of hybridization complexes formed for each nucleic acid target. Such means can be any suitable for defining the classification rules for use of the biomarkers of the invention in diagnosing UC or CD. Such classification rules can be generated via any suitable means known in the art, including but not limited to supervised or unsupervised classification techniques. In a preferred embodiment, classification rules are generated by use of supervised classification techniques. As used herein, “supervised classification” is a computer-implemented process through which each measurement vector is assigned to a class according to a specified decision rule, where the possible classes have been defined on the basis of representative training samples of known identity. Examples of such supervised classification include, but are not limited to, classification trees, neural networks, k-nearest neighbor algorithms, linear discriminant analysis (LDA), quadratic discriminant analysis (QDA), and support vector machines.

In one non-limiting example, a weighted combination of the genes is arrived at by, for example, a supervised classification technique which uses the expression data from all of the genes within individual patients. The expression level of each gene in a patient is multiplied by the weighting factor for that gene, and those weighted values for each gene's expression are summed for each individual patient, and, optionally, a separate coefficient specific for that comparison is added to the sum which gives a final score. Each comparison set may result in its own specific set of gene weightings; for example, an IBD v Normal may utilize different gene expression weightings than CD v UC. Weightings can also have either a positive-sign or a negative-sign. Not all patients in one classification will have the same Gene 1 up, Gene 2 down, etc. (See examples below).

In various embodiments of this third aspect of the invention, the two or more probe sets comprise or consist of at least 3, 4, 5, 6, 7, 8, 9, or 10 probe sets, and wherein none of the 3-10 probe sets selectively hybridize to the same nucleic acid target. These embodiments of probe sets are further discussed is the first and second aspects of the invention; ail other embodiments of the probe sets and polynucleotides of the first and second aspect can be used in the methods of the invention.

In a first preferred embodiment of this third aspect of the invention, the methods comprise use of a first probe set that selectively hybridizes under high stringency conditions to CB4 (SEQ ID NO:5), or a full complement thereof, a second probe set that selectively hybridizes under high stringency conditions to DNAJA1 (SEQ ID NO:6), or a full complement thereof, and a third probe set that selectively hybridizes under high stringency conditions to MMD (SEQ ID NO:2), or a full complement thereof. As disclosed in more detail below, the inventors have discovered that such methods can be used to distinguish between UC and CD patients.

In a second specific embodiment of this third aspect of the invention, the methods comprise use of a first probe set that selectively hybridizes under high stringency conditions to MMD (SEQ ID NO:2), or a full complement thereof, a second probe set that selectively hybridizes under high stringency conditions to PDIA6 (SEQ ID NO:4), or a full complement thereof, a third probe set that selectively hybridizes under high stringency conditions to QARS (SEQ ID NO:9), or a full complement thereof, and a fourth probe set that selectively hybridizes under high stringency conditions to WIPF1 (SEQ ID NO:10), or a full complement thereof. As disclosed is more detail below, the inventors haw discovered that such methods can be used to distinguish between UC and CD patients.

In a third specific embodiment of this third aspect of the invention, the methods comprise use of a first probe set that selectively hybridizes under high stringency conditions to MMD (SEQ ID NO:2), or a full complement thereof; a second probe set that selectively hybridizes under high stringency conditions to PDIA6 (SEQ ID NO:4) or a full complement thereof; a third probe set that selectively hybridizes under high stringency conditions to QARS (SEQ ID NO:9), or a full complement thereof; and a fourth probe set that selectively hybridizes under high stringency conditions to one or more of SEQ ID NO:11, SEQ ID NO:12, SEQ ID NO:13, SEQ ID NO:14, SEQ ID NO:15, and/or SEQ ID NO:16 (IGH), or full complements thereof. As disclosed in more detail below, the inventors have discovered that such methods can be used to distinguish between UC and CD patients. In one preferred embodiment, the fourth probe set selectively hybridizes under high stringency conditions to one or more of SEQ ID NO:11 (IGHG3); in another preferred embodiment, the fourth probe set selectively hybridizes to each of SEQ ID NO:11, SEQ ID NO:12, SEQ ID NO:13, SEQ ID NO:14, SEQ ID NO:15, and SEQ ID NO:16 (IGH). In a fourth specific embodiment of this third aspect of the invention, the methods comprise use of a first probe set that selectively hybridizes under high stringency conditions to CD4 (SEQ ID NO:5), or a full complement thereof, a second probe set that selectively hybridizes under high stringency conditions to DNAJA1 (SEQ ID NO:6), or a full complement thereof, a third probe set that selectively hybridizes under high stringency conditions to MMD (SEQ ID NO:2), or a full complement thereof, a fourth probe set that selectively hybridizes under high stringency conditions to RBM4(SEQ ID NO:8), or a full complement thereof, and a fifth probe set that selectively hybridizes under high stringency conditions to WIPF1 (SEQ ID NO:10), or a full complement thereof As disclosed in more detail below, the inventors have discovered that such methods can be used to distinguish between UC and CD patients.

In a fourth aspect, the present invention provides methods method for diagnosing UC or CD comprising:

(a) contacting a mRNA-derived nucleic acid sample obtained from a subject suspected of having UC or CD under amplifying conditions with 2 or more primer pairs, wherein at least a first primer pair and a second primer pair are capable of selectively amplifying a detectable portion of a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ED NO:8), QARS (SEQ ID NO:9), and WIPF1 (SEQ ID NO: 10), or full complements thereof; wherein the first primer pair and the second primer pair do not selectively amplify the same nucleic acid target;

(b) detecting amplification products generated by amplification of nucleic acid targets in the nucleic acid sample by the two or more primer pairs, wherein the amplification products provide a measure of gene expression of the nucleic acid targets; and

(c) diagnosing whether the subject is likely to nave UC or CD based on the amplification of the nucleic acid targets.

Definitions of primer pairs as used above apply to this aspect of the invention, as well as all other common terms, such as the relevant subject class. All embodiments disclosed above for the other aspects of the invention are also suitable for this fourth aspect.

In a preferred embodiment of the fourth aspect of the invention, the subject has a diagnosis of IBD, and the method thus comprises distinguishing whether the subject has UC or CD.

In these methods, amplification of target nucleic acids using the primer pairs is used instead of hybridization to detect gene expression products. Any suitable amplification technique can be used, including but not limited to PCR, RT-PCR, qPCR, qRT-PCR, qRT-PCR, spPCR, etc. Suitable amplification conditions can be determined by those of skill in the art based on the particular primer pair design and other factors, based on the teachings herein. In various embodiments, the two or more primer pairs comprise at least 3-10 primer pairs, wherein none of the 3-10 primer pairs selectively amplify the same nucleic acid.

The methods comprise either (a) diagnosing whether the subject is likely to have UC or CD; or (b) distinguishing whether a subject with an established diagnosis of IBD has UC or CD, based on the gene expression of the nucleic acid targets. As used herein, “likely to have” means a statistically significant likelihood that the diagnosis is correct. In various embodiments, the method results in an accurate diagnosis in at least 70% of cases; more preferably of at least 75%, 80%, 85%, 90%, or more of the cases.

In a first preferred embodiment the methods comprise use of a first primer pair capable of selectively amplifying a detectable portion of CD4 (SEQ ID NO: 5), or a full complement thereof, a second primer pair capable of selectively amplifying a detectable portion of DNAJA1 (SEQ ID NO: 6), or a full complement thereof, and a third primer pair capable of selectively amplifying a detectable portion of MMD (SEQ ED NO:2), or a full complement thereof. As disclosed is more detail below, the inventors have discovered that such methods are particularly useful to distinguish between UC and CD patients. to a second preferred embodiment of this fourth aspect of the invention, the methods comprise use of a first primer pair capable of selectively amplifying a detectable portion of MMD (SEQ ID NO:2) or a full complement thereof; a second primer pair capable of selectively amplifying a detectable portion of PDIA6 (SEQ ID NO:4) or a full complement thereof; a third primer pair capable of selectively amplifying a detectable portion of QARS (SEQ ID NO:9) or a full complement thereof; and a fourth primer pair capable of selectively amplifying a detectable portion of WIPF1 (SEQ ID NO: 10) or a full complement thereof As disclosed in more detail below, the inventors have discovered that such biomarkers are particularly useful as probes to distinguish between UC and CD patients.

In a third preferred embodiment, the methods comprise use of a first primer pair mat selectively amplifies a detectable portion of MMD (SEQ ID NO: 2) or a full complement thereof; a second primer pair that selectively amplifies a detectable portion of PDIA6 (SEQ ID NO:4) or a fell complement thereof; a third primer pair that selectively amplifies a detectable portion of QARS (SEQ ID NO:9) or a full complement thereof; and a fourth primer pair that selectively amplifies a detectable portion of one or more of SEQ ID NO:11, SEQ ID NO:12, SEQ ID NO:13, SEQ ID NO:14, SEQ ID NO:15, and/or SEQ ID NO:16 (IGH), or full complements thereof. As disclosed in more detail below, the inventors have discovered that such methods are particularly useful to distinguish between UC and CD patients. In one preferred embodiment, the fourth primer pair selectively amplifies SEQ ID NO:11 (IGHG3). In another preferred embodiment, the fourth primer pair selectively amplifies each of SEQ ID NO:11, SEQ ID NO:12, SEQ ID NO:13, SEQ ID NO:14, SEQ ID NO:15, and SEQ ID NO:16.

In a fourth preferred embodiment the methods comprise use of a first primer pair that selectively amplifies a detectable portion of CD4 (SEQ ID NO:5), or a full complement thereof, a second primer pair that selectively amplifies a detectable portion of DNAJA1 (SEQ ID NO:6), or a full complement thereof, a third primer pair that selectively amplifies a detectable portion of MMD (SEQ ID NO:2), or a full complement thereof, a fourth primer pair that selectively amplifies a detectable portion of RBM4 (SEQ ID NO:8), or a full complement thereof, and a fifth primer pair that selectively amplifies a detectable portion of WIPF1 (SEQ ID NO:10), or a full complement thereof. As disclosed in mere detail below, the inventors have discovered that such methods can be used to distinguish between UC and CD patients.

In various embodiments, the methods may further comprise comparing amplification products to a control.

In a further embodiment of ail of the methods of the invention, the methods are automated, and appropriate software is used to conduct some or all stages of the method.

In a fifth aspect the present invention provides methods for diagnosing IBD comprising:

(a) contacting a mRNA-derived nucleic acid sample obtained from a subject suspected of having IBD under hybridizing conditions with 2 or more probes sets, wherein at least a first probe set and a second probe set selectively hybridize under high stringency conditions to a nucleic acid target selected from the group consisting of IGH (SEQ ID NO: 11,12, 13,14,15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO: 8), QARS (SEQ ID NO:9), and WIPF1 (SEQ ID NO:10), or full complements thereof; wherein, the first probe set and the second probe set do not selectively hybridize to the same nucleic acid target;

(b) detecting formation of hybridization complexes between the 2 or more probe sets and nucleic acid targets in the nucleic acid sample, wherein a number of such hybridization complexes provides a measure of gene expression of the nucleic acid targets; and

(c) diagnosing whether the subject is likely to have IBD based on the gene expression of the nucleic acid targets.

The inventors have discovered that the methods of the invention can be used, for example, in diagnosing and distinguishing IBD patients from normal patients. The specific genes, probe sets, hybridizing conditions, probe types, polynucleotides, etc. are as defined above for the first and/or second aspects of the invention.

The subject is any human subject that may be suffering from IBD. Symptoms of IBD include, but are not limited to, abdominal pain, constipation and/or diarrhea, and/or a change in bowel habits, vomiting, hematochezia, weight, loss, and/or weight gain; thus, for example, subjects with one or more of these symptoms would be candidate subjects for the methods of the invention.

All common terms used in this fifth aspect have the same meaning as used in other aspects. In a preferred embodiment, “mRNA-derived nucleic acid sample” is from any suitable tissue source, including but not limited to Mood samples, such as PBMCs, SBC-depleted whole blood, or lysed whole blood.

In one preferred embodiment, the mRNA sample is a human mRNA sample. It will be understood by those of skill in the art that the RNA sample does not require isolation of an individual or several individual species of RNA molecules, as a complex sample mixture containing RNA to be tested can be used, such as a cell or tissue sample analyzed by in situ hybridization.

In a further embodiment, the probe sets comprise single stranded anti-sense polynucleotides of the nucleic acid compositions of the invention. For example, in mRNA fluorescence in situ hybridization (FISH) (i.e. FISH to detect messenger RNA), only an anti-sense probe strand hybridizes to the single stranded mRNA in the RNA sample, and in that embodiment, the “sense” strand oligonucleotide can be used as a negative control.

Alternatively, the probe sets may comprise DNA probes. In either of these embodiments (anti-sense probes or cDNA probes), it is preferable to use controls or processes that direct hybridization to either cytoplasmic mRNA or nuclear DNA. In the absence of directed hybridization, it is preferable to distinguish between hybridization to cytoplasmic RNA and hybridization to nuclear DNA.

Any method for evaluating the presence or absence of hybridization products in the sample can be used, such as by Northern blotting methods, in situ hybridization (for example, on blood smears), polymerase chain reaction (PCR) analysis, qPCR (quantitative PCR), RT-PCR (Real Time PCR), qRT-PCR (quantitative RT-PCR), or array based methods.

In one embodiment, detection is performed by in situ hybridization (“ISH”), as disclosed above.

In a further embodiment, an array-based format can be used in which the probe sets can be arrayed on a surface and the RNA sample is hybridized to the polynucleotides on the surface, as disclosed above.

In each of the above aspects and embodiments, detection of hybridization is typically accomplished through the use of a detectable label on the polynucleotides in the probe sets, such as those described above; in some alternatives, the label can be on the target nucleic acids. The label can be directly incorporated into the polynucleotide, or if can be attached to a probe or antibody which hybridizes or binds to the polynucleotide. The labels may be coupled to the probes in a variety of means known to those of skill is the art, as described above. The label can be detected by any suitable technique, including but not limited to spectroscopic, photochemical, biochemical, immunochemical, physical, or chemical techniques, as discussed above.

The methods may comprise comparing gene expression of the nucleic acid targets to a control. Any suitable control knows in the art can be used is the methods of the invention. For example, the expression level of a gene known to be expressed at a relatively constant level in IBD and normal patients can be used for comparison. Alternatively, the expression level of the genes targeted by the probes can be analyzed in normal RNA samples equivalent to the test sample. Another embodiment is the use of a standard concentration curve that gives absolute copy numbers of the mRNA of the gene being assayed; this might obviate the need for a normalization control because the expression levels would be given in terms of standard concentration units. Those of skill in the art will recognize that many such controls can be used in the methods of the invention.

The methods comprise diagnosing whether the subject is likely to have IBD based on the gene expression of the nucleic acid targets. As used herein, “likely to have” means a statistically significant likelihood that the diagnosis is correct. In various embodiments, the method results in an accurate diagnosis is at least 70% of cases; more preferably of at least 75%, 80%, 85%, 90%, or more of the cases.

The methods of the present invention may apply weights, derived by various means in the art, to the number of hybridization complexes formed for each nucleic acid target. Such means can be any suitable for defining the classification rules for use of the biomarkers of the invention in diagnosing IBD. Such classification rules can be generated via any suitable means known in the art, including but not limited to supervised or unsupervised classification techniques. In a preferred embodiment, classification roles are generated by use of supervised classification techniques. As used herein, “supervised classification” is a computer-implemented process through which each measurement vector is assigned to a class according to a specified decision rule, where the possible classes have been defined on the basis of representative training samples of known identity. Examples of such supervised classification include, but are not limited to, classification trees, neural networks, k-nearest neighbor algorithms, linear discriminant analysis (LDA), quadratic discriminant analysis (QDA), and support vector machines.

In one non-limiting example, a weighted combination of the genes is arrived at by, for example, a supervised classification technique which uses the expression data from all of the genes within individual patients. The expression level of each gene in a patient is multiplied by the weighting factor for that gene, and those weighted values for each gene's expression are summed for each individual patient, and, optionally, a separate coefficient specific for that comparison, is added to the sum which gives a final score. Weightings can also have either a positive-sign or a negative-sign. Not all patients in one classification will haw the same Gene 1 up, Gene 2 down, etc. (See examples below).

In various embodiments of this fifth aspect of the invention, the two or more probe sets comprise or consist of at least 3, 4, 5, 6, 7, 8, 9, or 10 probe sets, and wherein none of the 3-10 probe sets selectively hybridize to the same nucleic acid target. These embodiments of probe sets are further discussed in the first and second aspects of the invention; all other embodiments of the probe sets and polynucleotides of the first and second aspect can be used in the methods of the invention.

Is a first preferred embodiment of this fifth aspect of the invention, the methods comprise use of a first probe set that selectively hybridizes under high stringency conditions to MMD (SEQ ID NO:2), or a fall complement thereof; a second probe set that selectively hybridizes under high, stringency conditions to PDIA6 (SEQ ID NO:4), or a full complement thereof; a third probe set that selectively hybridizes under high stringency conditions to QARS (SEQ ID NO:9), or a full complement thereof; and a fourth probe set that selectively hybridizes under high stringency conditions to one or more of SEQ ID NO:11, SEQ ID NO:12, SEQ ID NO:13, SEQ ID NO:14, SEQ ID NO:15, and/or SEQ ID NO:16 (IGH), or full complements thereof. As disclosed in more detail below, the investors have discovered that such methods can be used to distinguish between IBD and normal patients. In one preferred embodiment, the fourth probe set selectively hybridizes under high stringency conditions to one or more of SEQ ID NO:11(IGHG3); is another preferred embodiment, the fourth probe set selectively hybridizes to each of SEQ ID NO:11, SEQ ID NO:12, SEQ ID NO:13, SEQ ID NO:14, SEQ ID NO:15, and SEQ ID NO:16 (IGH).

In a second preferred embodiment of this fifth aspect of the invention, the methods comprise use of a first probe set that selectively hybridizes under high stringency conditions to PDLIM1 (SEQ ID NO:3), or a full complement thereof, a second probe set that selectively hybridizes under high stringency conditions to PDIA6 (SEQ ID NO:4), or a full complement thereof, a third probe set that selectively hybridizes under high stringency conditions to RBM4 (SEQ ID NO:8), or a full complement thereof, a fourth probe set that selectively hybridizes under high stringency conditions to QARS (SEQ ID NO:9 and a fifth probe set that selectively hybridizes under high stringency conditions to WIPF1 (SEQ ID NO: 10), or a full complement thereof. As disclosed in more detail below, the inventors have discovered that such methods can be used to distinguish between IBD and normal patients.

In a third preferred embodiment of this fifth aspect of the invention, the methods comprise use of a first probe set that selectively hybridizes under high stringency conditions to CD4 (SEQ ID NO:5), or a full complement thereof, a second probe set mat selectively hybridizes under high stringency conditions to PDLIM1 (SEQ ID NO:3), or a full complement thereof, and a third probe set that selectively hybridizes under high stringency conditions to RBM4 (SEQ ID NO:8), or a full complement thereof As disclosed is more detail below, the inventors have discovered that such methods can be used for diagnosing IBD.

In a sixth aspect, the present invention provides methods method for diagnosing IBD comprising:

(a) contacting a mRNA-derived nucleic acid sample obtained from a subject suspected of having IBD under amplifying conditions with 2 or more primer pairs, wherein at least a first printer pair and a second primer pair are capable of selectively amplifying a detectable portion of a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO: 10), or full complements thereof; wherein the first primer pair and the second primer pair do not selectively amplify the same nucleic acid target;

(b) detecting amplification products generated by amplification of nucleic acid targets in the nucleic acid sample by the two or more primer pairs, wherein the amplification products provide a measure of gene expression of the nucleic acid targets; and

(c) diagnosing whether the subject is likely to have IBD based on the amplification of the nucleic acid targets.

Definitions of primer pairs as used above apply to tins aspect of the invention, as well as all other common terms. All embodiments disclosed above for the other aspects of the invention are also suitable for this sixth aspect.

In these methods, amplification of target nucleic acids using the primer pairs is used instead of hybridization to detect gene expression products. Any suitable amplification technique can be used, including but not limited to PCR, PT-PCR, qPCR, qRT-PCR, spPCR, etc. Suitable amplification conditions can be determined by those of skill in the art based on the particular primer pair design and other factors, based on the teachings herein. In various embodiments, the two or more primer pairs comprise at least 3-10 primer pairs, wherein none of the 3-10 primer pairs selectively amplify the same nucleic acid.

In a first preferred embodiment, the methods comprise use of first primer pair that selectively amplifies a detectable portion of MMD (SEQ ID NO:2) or a full complement thereof; a second primer pair that selectively amplifies a detectable portion of PDIA6(SEQ ID NO:4) or a full complement thereof; a third primer pair mat selectively amplifies a detectable portion of QARS (SEQ ID NO:9) or a full complement thereof; and a fourth primer pair that selectively amplifies a detectable portion of one or more of SEQ ID NO:11, SEQ ID NO:12, SEQ ID NO:13, SEQ ID NO:14, SEQ ID NO:15, and/or SEQ ID NO:16 (IGH), or full complements thereof. As disclosed in more detail below, the inventors have discovered that such methods are particularly useful to distinguish between IBD and normal patients. In one preferred embodiment, the fourth primer pair selectively amplifies SEQ ID NO:11 (IGHG3). In another preferred embodiment, the fourth primer pair selectively amplifies each of SEQ ID NO:11, SEQ ID NO:12, SEQ ID NO:13, SEQ ID NO:14, SEQ ID NO:15, and SEQ ID NO: 16.

In a second preferred embodiment of this aspect of the invention, the methods comprise use of a first primer pair capable of selectively amplifying a detectable portion of PDLIM1 (SEQ ID NO:3), or a full complement thereof, a second primer pair capable of selectively amplifying a detectable portion of PDIA6 (SEQ ID NO:4), or a full complement thereof, a third primer pair capable of selectively amplifying a detectable portion of RBM4 (SEQ ID NO:8), or a full complement, thereof, a fourth primer pair capable of selectively amplifying a detectable portion of QARS (SEQ ID NO:9 and a fifth primer pair capable of selectively amplifying a detectable portion of WIPF1 (SEQ ID NO:10), or a full complement thereof. As disclosed in more detail below, the inventors have discovered that such methods can be used to distinguish between IBD and normal patients.

In a third preferred embodiment of this aspect of the invention, the methods comprise use of a first primer pair capable of selectively amplifying a detectable portion of CD4 (SEQ ID NO:5), or a full complement thereof, a second primer pair capable of selectively amplifying a detectable portion of PDLIM1 (SEQ ID NO:3), or a full complement thereof, and a third primer pair capable of selectively amplifying a detectable portion of RBM4 (SEQ ID NO:8), or a full complement thereof. As disclosed in more detail below, the inventors have discovered that such methods can be used for diagnosing IBD.

In various embodiments, the methods may further comprise comparing amplification products to a control. In a seventh aspect, the present invention provides methods for diagnosing IBD and providing a differential diagnosis of UC or CD comprising:

(a) contacting a mRNA-derived nucleic acid sample obtained from a subject suspected of having IBD under hybridizing conditions with 2 or more probes sets, wherein at least a first probe set and a second probe set selectively hybridize under high stringency conditions to a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO: 6), HBA2(SEQ ID NO:7), RBM4 (SEQ ID NO:8), QARS (SEQ ID NO:9), and WIPF1 (SEQ ID NO:10), or fell complement thereof; wherein the first probe set and the second probe set do not selectively hybridize to the same nucleic acid target;

(b) detecting formation of hybridization complexes between the 2 or more probe sets and nucleic acid targets in the nucleic acid sample, wherein a number of such hybridization complexes provides a measure of gene expression of the nucleic acid targets; and

(c) diagnosing whether the subject is likely to have IBD based on the gene expression of the nucleic acid targets

(d) further diagnosing whether the IBD patient has UC or CD based on the gene expression of the nucleic acid targets

In an eighth aspect, the present invention provides methods for diagnosing IBD and providing a differential diagnosis of UC or CD comprising:

(a) contacting a mRNA-derived nucleic acid sample obtained from a subject suspected of having IBD under amplifying conditions with 2 or more primer pairs, wherein at least a first primer pair and a second primer pair are capable of selectively amplifying a detectable portion of a nucleic acid target selected from the group consisting of IGH (SEQ ID NO: 11, 12, 13, 14, 15, and/or 16), MMD (SEQ ID NO: 2), PDLIM1 (SEQ ED NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1. (SEQ ID NO:10), or full complements thereof; wherein the first primer pair and the second primer pair do not selectively amplify the same nucleic acid target;

(b) detecting amplification products generated by amplification of nucleic acid targets in the nucleic acid sample by the two or more primer pairs, wherein the amplification products provide a measure of gene expression of the nucleic acid targets; and

(c) diagnosing whether the subject is likely to have JED based on the amplification of the nucleic acid targets; and

(d) further diagnosing whether the IBD patient, has UC or CD based on the amplification of the nucleic acid targets.

The inventors have discovered that the methods of the invention can be used, for example, hi diagnosing and distinguishing IBD patients from normal patients and further providing a differential diagnosis of UC or CD when the patient has a confirmed diagnosis of IBD. The specific genes, probe sets, hybridizing conditions, probe types, polynucleotides, etc. are as defined above for the first and/or second aspects of the invention.

The subject is any human subject mat may be suffering from IBD. Definitions of terms in the seventh and eighth aspects are as used in previous aspects of the invention, as well as all other common terms. All embodiments disclosed above for the other aspects of the invention are also suitable for the seventh and eighth aspects.

In this aspect, any of the embodiments of the fifth and sixth aspects of the invention for diagnosing IBD are used in combination with any embodiments of the third and fourth aspects of the invention for distinguishing UC or CD. In one preferred embodiment, any of the embodiments above for diagnosing IBD are carried out simultaneously with any of the embodiments above for distinguishing UC from CD. This preferred embodiment permits improved efficiency and accuracy is carrying out all gene expression analyses simultaneously. In a further preferred embodiment, any embodiment above for diagnosing IBD is carried out, and those samples diagnosed as IBD are then assayed using any embodiments of the fifth and sixth aspects of the invention. This embodiment provides for reduced costs by distinguishing UC from CD only in IBD-positive samples. This embodiment is preferably automated, so that an IBD-positive sample is automatically tested to distinguish UC from CD.

In various preferred embodiments of the seventh and eighth aspects, the methods comprise combining the methods of the invention as follows:

(a) Third aspect, first preferred embodiment+Fifth aspect, first preferred embodiment;

(b) Third aspect, first preferred, embodiment+Fifth aspect, second preferred embodiment;

(c) Third aspect, first preferred embodiment+Fifth aspect, third preferred embodiment;

(d) Third aspect, second preferred embodiment+Fifth aspect, first preferred embodiment;

(e) Third aspect, second preferred embodiment+ Fifth aspect second preferred embodiment;

(f) Third aspect, second preferred embodiment+Fifth aspect, third preferred embodiment;

(g) Third aspect, third preferred embodiment+Fifth aspect, first preferred embodiment;

(h) Third aspect, third preferred embodiment+Fifth aspect, second preferred embodiment;

(i) Third aspect, third preferred embodiment+Fifth aspect, third preferred embodiment;

(j) Third aspect, fourth preferred embodiment+Fifth aspect, first preferred embodiment;

(k) Third aspect, fourth preferred embodiment+Fifth aspect, second preferred embodiment;

(l) Third aspect, fourth preferred embodiment+Fifth aspect, third preferred embodiment;

(m) Fourth aspect, first preferred embodiment+Sixth aspect, first preferred embodiment;

(n) Fourth aspect first preferred embodiment+Sixth aspect, second preferred embodiment;

(o) Fourth aspect, first preferred embodiment+Sixth aspect, third preferred embodiment;

(p) Fourth aspect, second preferred embodiment+ Sixth aspect, first preferred embodiment;

(g) Fourth aspect, second preferred, embodiment+Sixth aspect, second preferred embodiment;

(r) Fourth aspect, second preferred embodiment+Sixth aspect, third preferred embodiment;

(s) Fourth aspect, third preferred embodiment+Sixth aspect, first preferred embodiment;

(t) Fourth aspect, third preferred embodiment+Sixth aspect, second preferred embodiment;

(u) Fourth aspect, third preferred embodiment+Sixth aspect, third preferred embodiment;

(v) Fourth aspect, fourth preferred embodiment+Sixth aspect, first preferred, embodiment;

(w) Fourth aspect, fourth preferred embodiment+Sixth aspect, second preferred embodiment; and

(x) Fourth aspect fourth preferred embodiment+Sixth aspect third preferred embodiment. In a preferred embodiment of all of the embodiments of the third, fourth, fifth, sixth, seventh, and eighth aspects of the invention, the methods further comprise making a treatment decision based on the diagnosis or distinguishing accomplished by the methods. In this embodiment, an attending physician considers the results of the methods in combination with other clinical factors in determining a specific course of treatment for the subject. As noted above, treatment regimens for UC and CD are distinct, and thus the results obtained, using the methods of the invention will comprise an important part of the factors on which an attending physician will make a treatment decision.

In a further embodiment of all of the methods of the invention, the methods are automated, and appropriate software is used to conduct some or all stages of the method. Thus, the present invention provides non-transitory computer readable storage media, for automatically carrying out the methods of any aspect/embodiment of the invention on a gene expression detection device, including but not limited to those disclosed below. As used herein the term “computer readable medium” includes magnetic disks, optical disks, organic memory, and any other volatile (e.g. Random Access Memory (“RAM”)) or non-volatile (e.g., Read-Only Memory (“ROM”)) mass storage system readable by the CPU. The computer readable medium includes cooperating or interconnected computer readable medium, which exist exclusively on the processing system or be distributed among multiple interconnected processing systems that may be local or remote to the processing system.

In a further aspect, the present invention provides kits for use in the methods of the invention, comprising the biomarkers and/or primer pair sets of the invention and instructions for their use. In a preferred embodiment, the polynucleotides are detectably labeled, most preferably where the detectable labels on each polynucleotide is a given probe set or primer pair are the same, and differ from, the detectable labels on the polynucleotides in other probe sets or primer pans, as disclosed above. In a further preferred embodiment, the probes/primer pairs are provided in solution, most preferably in a hybridization or amplification buffer to be used in the methods of the invention. In further embodiments, the kit also composes wash solutions, pre-hybridization solutions, amplification reagents, software for automation of the methods, etc.

### EXAMPLE 1

In an effort to identify gene expression profiles that could discriminate between whole blood samples collected from UC, and CD patients, and thus provide the basis for a minimally invasive diagnostic test, we employed a proprietary data, mining program to analyze publicly available data collected from Crohn's Disease (CD) patients. Ulcerative Colitis (UC) patients, and healthy individuals (Burczynski et al., Molecular Classification of Crohn's Disease and Ulcerative Colitis Patients Using Transcriptional Profiles in Peripheral Blood Mononuclear Cells, Journal of Molecular Diagnostics 8 (1): 51-61, February 2006), hereinafter referred to as the “Burczynski data.”

The Burczynski data consisted of a set of individual expression level features, each feature being a quantitative fluorescent signal derived from a single microarray spot. As detailed in Burczynski et al (2006), the signals were generated by hybridizing fluorescently-labeled RNA from a whole blood sample collected from a single patient to all of the spots on a single DNA-based oligonucleotide microarray. From these data, we identified molecular signatures, comprised of sets of expression level features, that effectively differentiated between IBD patients and unaffected normal control subjects. Expression levels of the genes represented by those array features were then measured in a prospectively ascertained sample of patients (the ‘pilot study’, described below). The Burczynski discovery dataset consisted of 127 separate Affymetrix microarray hybridization experiments on RNA from 26 Ulcerative Colitis patients, 59 Crohn's Disease patients, and 42 normal controls.

We employed oar proprietary data mining program to analyze the publicly available Burczynski data. We randomly divided the patients in the Burczynski dataset for purposes of our analysis into 2 approximately equal groups: a training set for biomarker set discovery and a separate non-overlapping test set for assessment of biomarkers discovered from the training set. The training set consisted of 28 CD patients and 15 UC patients. The test set consisted of 31 CD patients and 11 UC patients. The CD patients were defined as ‘CD’ and UC patients were defined as ‘UC’.

Our proprietary data mining program was then used to perform a genetic-algorithm search of expression level data for combinations across the CD and UC patient sets. The number of features constituting a marker set combination was fixed at 4. The Burczynski data set contained 22,283 expression level features; the number of 4-wise combinations of features in that data set are:

(122,283/(14*122,279)=10,265,905,646,716,170.

The proprietary data mining program was run 3 separate times on the Burczynski dataset using three specific sets of parameters. (1) One parameter set used the training and test sets defined above with additional settings that gave computational results weighted towards higher sensitivity for UC (to minimize false negatives), (2) the second set was similarly weighted towards higher specificity for UC (to minimize false positives), and (3) the third set used random cross-validation (‘bootstrap’) with no weighting towards either specificity or sensitivity. Each 4-feature combination analyzed was assigned a score that characterized its accuracy in discriminating between the affected and unaffected groups. The score for each combination of expression features ranges from 1.00 for completely accurate to 0.00 for completely inaccurate.

For the set weighted towards higher sensitivity (set 1), the top-scoring 4-feature sets were obtained such that a combination's score on the training set was greater than 0.99, or the combination's score on the test set was greater than 0.90. For the set weighted towards higher specificity (set 2), the top-scoring 4-feature sets were obtained such that a combination's score on the training set was greater than 0.99, or the combination's score on the test set was greater than 0.90. For the bootstrap result set with equal weighting between sensitivity and specificity (set 3), the top-scoring 4-feature sets were obtained such that a combination's score on the training set was greater than 0.94 (i.e.: approximately 94% accuracy).

Significance of the marker sets was assessed empirically by random iterative relabeling. The affected and unaffected statuses of patients were randomly re-assigned, and the proprietary data mining program was then run to determine the top marker solutions for the randomly labeled set. This was repeated to obtain 100,000 marker sets. In the randomly relabeled sets, for set 1 weighted towards higher sensitivity as above, the training scores reached a maximum of 0.983; 95% of solutions (the empirical p=0.05 level) scored at or below 0.924, and 99% of solutions the empirical p=0.01 level) scored at or below 0.941. The test set scores in relabeled solutions of set 1 reached a maximum of 0.885; 95% of solutions (the empirical p=0.05 level) scored at or below 0.719, and 99% of solutions (the empirical p=0.01 level) scored at or below 0.763.

A total of fifteen sets, each comprised of 4 features (in the Burczynski microarray data, some genes are represented by more than 1 feature and some features hybridize to more than 1 gene), was obtained using a combination of thresholds: the score on the training set was greater than 0.99 and/or the score on the test set was greater than 0.90 and/or the bootstrap score was greater than 0.94.

Table 2 contains the gene combinations that effectively differentiate between UC and CD patients. These 4-gene combinations were identified from the gene expression profile of peripheral blood mononuclear cells of UC and CD patients.

TABLE 2
Gene Combinations differentiating UC and CD

| Combination | Gene 1 | Gene 2 | Gene 3 | Gene 4 |
|---|---|---|---|---|
| Combination 1 | IGH | PDLIM1 | MAFF | CTBP1 |
| Combination 2 | IGH | PDLIM1 | CD4 | QARS |
| Combination 3 | IGH | PDLIM1 | WASPIP | TMEM109 |
| Combination 4 | IGH | PDLIM1 | CD4 | RBM4 |
| Combination 5 | IGH | MMD | PDLIM1 | PDIA6 |
| Combination 6 | IGH | MMD | PDLIM1 | CD4 |
| Combination 7 | IGH | CD4 | RAVER2 | GNG11 |
| Combination 8 | IGH | MMD | PDLIM1 | MAFF |
| Combination 9 | IGH | MMD | WASPIP | TMEM109 |
| Combination 10 | IGH | MMD | WASPIP | HBA2 |
| Combination 11 | IGH | MMD | MAFF | DNAJA1 |
| Combination 12 | IGH | MMD | DNAJA1 | RBM4 |
| Combination 13 | IGH | MMD | DNAJA1 | RAVER2 |
| Combination 14 | IGH | MMD | MAFF | PDIA6 |
| Combination 15 | IGH | MMD | DNAJA1 | CTBP1 |

Fifteen individual expression array features constitute those 15 sets. The feature sets and their memberships are indicated in Table 3 below. Each feature represents a transcript from a single gene; the HUGO gene names for each feature are indicated. The average fold-difference in expression between the CD and UC groups is also shown, computed by dividing the average expression level is CD patients by the average expression level in UC patients. A fold-difference greater than 1 indicates the gene has higher expression in CD patients compared to UC patients, while a fold-difference less than 1 indicates the gene has lower expression in CD patients compared to UC patients. The row labeled “freq” shows how many times that microarray feature occurs in the top 15 marker sets.

TABLE 3
Feature sets and memberships for combinations differentiating UC and CD

| | HGNC name | | | | | | | |
|---|---|---|---|---|---|---|---|---|
| | IGH | MMD | PDLIM1 | MAFF | CD4 | DNAJA1 | PDIA6 | CTBP1 |
| affy | 211430_s_at | 203414_at | 208690_s_at | 36711_at | 203547_at | 200881_s_at | 207668_x_at | 203392_s_at |
| U133 # | | | | | | | | |
| CDvUC | 3.59 | 0.68 | 0.75 | 0.59 | 1.39 | 0.86 | 1.3 | 1.24 |
| fold | | | | | | | | |
| freq | 15 | 10 | 7 | 4 | 4 | 4 | 2 | 2 |
| sets | x | | x | x | | | | x |
| | x | | x | | x | | | |
| | x | | x | | | | | |
| | x | | x | | x | | | |
| | x | x | x | | | | x | |
| | x | x | x | | x | | | |
| | x | | | | x | | | |
| | x | x | x | x | | | | |
| | x | x | | | | | | |
| | x | x | | | | | | |
| | x | x | | x | | x | | |
| | x | x | | | | x | | |
| | x | x | | | | x | | |
| | x | x | | x | | | x | |
| | x | x | | | | x | | x |

| | HGNC name | | | | | | |
|---|---|---|---|---|---|---|---|
| | WASPIP | RAVER2 | TMEM109 | RBM4 | QARS | HBA2 | GNG11 |
| affy | 202664_at | 201648_at | 201361_at | 200997_at | 217846_at | 217414_x_at | 204115_at |
| U133 # | | | | | | | |
| CDvUC | 2.23 | 3 | 3.3 | 2.4 | 2.59 | 2.36 | 1.75 |
| fold | | | | | | | |
| freq | 3 | 2 | 2 | 2 | 1 | 1 | 1 |
| sets | | | | | x | | |
| | x | | x | | | | |
| | | | | x | | | |
| | | x | | | | | x |
| | x | | x | | | | |
| | x | | | | | x | |
| | | | | x | | | |
| | | x | | | | | |

A gene list (Table 4) was derived from an analysis (Table 3) of the unique set of genes in these combinations.

TABLE 4
Gene List

| HGNC | | | GenBank | |
|---|---|---|---|---|
| gene | Chromosome | NCBI Reference | Accession | |
| symbol | Location | Sequence | Number | HGNC Gene Name |
| IGHG3 | 14q32.33 | NG_001019.5 | M87789.1 | immunoglobulin heavy constant gamma 3 |
| | | (SEQ ID NO: 1A) | | (G3m marker) |
| IGHG1 | 14q32.33 | NG_001019.5 | BC067091.1 | immunoglobulin heavy constant gamma 1 |
| | | (SEQ ID NO: 1B) | | (G1m marker) |
| IGHM | 14q32.33 | NG_001019.5 | BC016381.1 | immunoglobulin heavy constant mu |
| | | (SEQ ID NO: 1C) | | |
| IGH@ | 14q32.33 | NG_001019.5 | BC073766.1 | immunoglobulin heavy locus |
| | | (SEQ ID NO: 1D) | | |
| IGHV4- | 14q32.33 | NG_001019.5 | BC073773.1 | immunoglobulin heavy variable 4-31 |
| 31 | | (SEQ ID NO: 1E) | | |
| IGHG4 | 14q32.33 | NG_001019.5 | BC025985.1 | immunoglobulin heavy constant gamma 4 |
| | | (SEQ ID NO: 1F) | | (G4m marker) |
| MMD | 17q | NM_012329.2 | — | monocyte to macrophage differentiation- |
| | | (SEQ ID NO: 2) | | associated |
| PDLIM1 | 10q23.1 | NM_020992.2 | — | PDZ and LIM domain 1 |
| | | (SEQ ID NO: 3) | | |
| PDIA6 | 2p25.1 | NM_005742.2 | — | protein disulfide isomerase family A, member 6 |
| | | (SEQ ID NO: 4) | | |
| CD4 | 12pter-p12 | NM_000616.3 | — | CD4 molecule |
| | | (SEQ ID NO: 5) | | |
| DNAJA1 | 9p13.3 | NM_001539.2 | — | DnaJ (Hsp40) homolog, subfamily A, member 1 |
| | | (SEQ ID NO: 6) | | |
| HBA2 | 16p13.3 | NM_000517.4 | — | hemoglobin, alpha 2 |
| | | (SEQ ID NO: 7) | | |
| RBM4 | 11q13 | NM_002896.2 | — | RNA binding motif protein 4 |
| | | (SEQ ID NO: 8) | | |
| QARS | 3p21.31 | NM_005051.1 | — | glutaminyl-tRNA synthetase |
| | | (SEQ ID NO: 9) | | |
| WIPF1 | 2q31.2 | NM_001077269.1 | — | WAS/WASL interacting protein family, |
| | | (SEQ ID NO: 10) | | member 1 |

### EXAMPLE 2

In a subsequent study, the genes shown in Table 4 were evaluated on RBC-depleted whole blood samples obtained from a new set of patients: 36 normal controls, 95 Ulcerative Colitis patients, and 97 Crohn's Disease patients. Samples were obtained from 7 clinical sites at various geographic locations within the U.S.A.

The RNA expression levels of the genes were measured by quantitative real-time PCR in the RBC-depleted whole Mood of the prospectively ascertained sample of affected patients and unaffected controls. The sequence similarity between the first six genes on the list allowed all six genes to be tested simultaneously as a single “IGH” gene using a single primer set and the data treated as if from a single gene. The relative expression levels of each gene were extrapolated from a standard curve created for each gene from a control sample diluted to known concentrations. Specifically, whole blood samples and clinical information were obtained from all patients. Each UC and CD patient was diagnosed by a board-certified gastroenterologist. All protocols were IRS approved; informed consent was obtained and peripheral blood samples and clinical data were collected from all patients. Expression data were obtained from peripheral whole blood samples (with no mononuclear enrichment) by isolating total mRNAs, synthesizing cDNAs, and performing real-time quantitative PCR on an Applied Biosystems 7300 Real-Time PCR System. Expression levels were output as Ct (cycle or crossing threshold). A standard curve was created for each gene by diluting a control sample to defined concentrations of 100, 1000, 10,000, and 200,000 ng/uL cDNA and assaying each concentration in the same reaction plate as the test samples. The quantities of each gene in the test samples were extrapolated from the standard curve. Each extrapolated gene quantity was then adjusted by the proportionate concentration of specimen cDNA relative to an arbitrarily selected cDNA concentration. The adjusted gene quantities were then converted to log2 and the log2(quantities) were used for analysis of diagnostic classification performance.

The 10 genes (considering all six IGH genes to be a single gene) include:
CD4
DNAJA1
HBA2
IGH
MMD
PDIA6
PDLIM1
QARS
RBM
WIPF1

While 9 of the 10 genes were found to be statistically significantly associated with UC or CD, and with differentiating IBD from normal subjects (Table 5), the individual genes are not highly accurate in discriminating the various subgroups. We investigated whether combinations of genes selected from the 10 might enable clinically useful marker accuracies.

TABLE 5
P-values for univariate associations of the genes with
UC v CD) and IBD vs normals

| probe | UC vs CD | IBD vs CONTROL |
|---|---|---|
| CD4 | 0.0015 | 1.68E−05 |
| DNAJA1 | 2.53E−14 | 0.0011 |
| HBA2 | 3.25E−07 | 0.8194 |
| IGH | 0.2362 | 7.90E−04 |
| MMD | 2.20E−16 | 5.97E−04 |
| PDIA6 | 2.13E−07 | 9.36E−01 |
| PDLIM1 | 7.99E−05 | 1.43E−05 |
| QARS | 6.89E−02 | 2.05E−03 |
| RBM4 | 2.53E−02 | 3.28E−09 |
| WIPF1 | 8.82E−05 | 9.49E−01 |

For each of the data subsets we evaluated the accuracy of gene combinations using logistic regression. The log2 (quantities) were analyzed using a reverse step wise logistic regression analysis to identify the best gene combinations for separating the UC from CD patients and the IBD from unaffected control subjects. One skilled in the art will understand that given a set of measurements, such as the gene expression values for a particular set of genes, and given these measurements across a particular set of samples, such as a group of UC samples mid a group of CD samples, there are many techniques for deriving from that data a ‘set of rules’ for classifying a sample as, e.g., UC or CD. Similarly, there are many techniques for deriving a set of rules for classifying a group of IBD patients as a distinct set of normal control subjects.

Those skilled in the art will understand that an algorithm, including a weighting for each gene expression level, will follow from the logistic regression analysis, according to one method of the body of knowledge known as ‘supervised learning’, which is a sub-field of ‘machine learning’, which itself can be considered sub-field of ‘data mining’. Supervised learning encompasses techniques for deriving algorithms, or rules, from data. One skilled in the art will understand that there are no clear boundaries between a standard statistical approach, and a ‘supervised learning’ approach, and that the classification formulas presented below could be considered as being derived from a supervised learning approach, but could also be termed a standard statistical approach. The logistic regression equations discovered from these analyses were adjusted to set the diagnostic threshold at zero. The logistic regression equations are used to calculate the expression indices.

Thus, the specific ups and downs of the expression levels of individual genes in the marker set do matter is the classifier, but not in a direct always-up or always-down manner. What matters is whether the sum of the weighted expression values is greater than or less man zero. A specific gene may have increased expression in one correctly classified patient, and that same gene may have a decreased expression in another correctly classified patient if the score is “compensated” by appropriately weighted changes in the expression of other genes in the marker set.

### 2A) UC vs CD 3-Gene Combination

One combination for separating the UC and CD patients was a three gene combination consisting of CD4, DNAJA1, and MMD.

The equation for calculating the UC/CD differential diagnostic expression index is:

3-gene UC v CD expression index=26.561.3+0.8398*log2(CD4)−1.0174*log2(DNAJA1)−1.2513*log2(MMD), where:

(CD4) is the quantity of CD4 extrapolated from the standard curve;

(DNAJA1) is the quantity of CD4 extrapolated from the standard curve; and

(MMD) is the quantity of CD4 extrapolated from the standard curve

TABLE 6a
UC vs CD (3-gene) Classification Matrix

| | CD | UC |
|---|---|---|
| test > 0 | 89 | 12 |
| test ≦ 0 | 8 | 83 |

An expression index greater man zero is diagnostic for CD and an index less man zero is diagnostic for UC.

TABLE 6b
Performance Measures

| | For | For |
|---|---|---|
| | UC | CD |
| Accuracy | 90% | 90% |
| Sensitivity | 87% | 92% |
| Specificity | 92% | 87% |
| Positive Predictive Value | 91% | 88% |
| Negative Predictive Value | 88% | 91% |

### 2B) UC vs CD 4-Gene Combination

A 4-gene combination, confuting MMD, PDIA6, QARS, and WIPF1, is also useful for a differential diagnosis of UC vs CD.

4-gene UC v CD expression index=25.3242−1.0916*log2(MMD)−0.5405*log2(PDIA6)+0.4912*log2(QARS)−0.2492*log2(WIPF1) where:

(MMD) is the quantity of MMD extrapolated from the standard curve;

(PDIA6) is the quantity of PDIA6 extrapolated from the standard curve;

(QARS) is the quantity of QARS extrapolated from the standard curve; and

(WIPF1) is the quantity of WIPF1 extrapolated from the standard curve

An expression index greater than zero is diagnostic for CD and an index less than zero is diagnostic for UC.

TABLE 7a
UC vs CD (4-gene) Classification Matrix

| | CD | UC |
|---|---|---|
| test > 0 | 84 | 18 |
| test ≦ 0 | 13 | 77 |

TABLE 7b
Performance Measures

| | For | For |
|---|---|---|
| | UC | CD |
| Accuracy | 84% | 84% |
| Sensitivity | 81% | 87% |
| Specificity | 87% | 81% |
| Positive Predictive Value | 86% | 82% |
| Negative Predictive Value | 82% | 86% |

### 2C) UC vs CD 4-Gene Combination

A second 4-gene combination, using IGHG3, MMD, PDIA6, and QARS is also useful for a differential diagnosis of UC vs CD.

4-gene UC v CD expression index=23.856+0.348*LOG2(IGHG3)−1.255*(LOG2(MMD)−0.736*(LOG2(PDIA6)+0.419*(LOG2(QARS)

(IGHG3) is the quantity of IGHG3 extrapolated from the standard curve;

(MMD) is the quantify of MMD extrapolated from the standard curve;

(PDIA6) is the quantity of PDIA6 extrapolated from the standard curve; and

(QARS) is the quantity of QARS extrapolated from the standard curve

An expression index greater man zero is diagnostic for CD and an index less than zero is diagnostic for UC.

TABLE 8a
UC vs CD (4-gene) Classification Matrix

| | CD | UC |
|---|---|---|
| test > 0 | 88 | 26 |
| test ≦ 0 | 9 | 67 |

TABLE 8b
Performance Measures

| | For | For |
|---|---|---|
| | UC | CD |
| Accuracy | 82% | 82% |
| Sensitivity | 72% | 91% |
| Specificity | 91% | 72% |
| Positive Predictive Value | 88% | 77% |
| Negative Predictive Value | 77% | 88% |

s2d) 4-Gene IBD vs Normal

A 4-gene combination, using IGHG3, MMD, PDIA6, and QARS is also useful for diagnosing IBD. This analysis included the 36 normal control patients and 192 IBD patients

4-gene IBD v normal expression index=10.7607−0.3508*log2(IGHG3)−0.7086*log2(MMD)+0.7542*log2(PDIA6)−0.1781*log2(QARS)

(IGHG3) is the quantity of IGHG3 extrapolated from the standard curve;

(MMD) is the quantify of MMD extrapolated from the standard curve;

(PDIA6) is the quantity of PDIA6 extrapolated from the standard curve; and

(QARS) is the quantity of QARS extrapolated from the standard curve

As expression index greater than zero is diagnostic for IBD and an index less than zero is not consistent with IBD.

TABLE 9a
IBD vs normal (4-gene) Classification Matrix

| | IBD | normal |
|---|---|---|
| test > 0 | 162 | 7 |
| test ≦ 0 | 28 | 29 |

TABLE 9b
Performance Measures

| | |
|---|---|
| Accuracy | 85% |
| Sensitivity | 85% |
| Specificity | 81% |
| Positive Predictive Value | 96% |
| Negative Predictive Value | 51% |

### 2e) 5-Gene IBD vs Normal

A 5-gene combination, using PDLIM1, PDIA6, RBM4, QARS, and WIPF1 is also useful for diagnosing IBD. This analysis included the 36 normal control patients and 192 IBD patients

b 6-gene IBD v Normal expression index=7.94860+1.4365*log2(PDIA6)−0.3465*log2(PDLIM1)−0.70908*log2(QARS)−0.81539*log2(RBM4)+0.33858*log2(WIPF1)

<0→IBD (IGHG3) is the quantity of IGHG3 extrapolated from the standard curve;

(PDIA6) is the quantity of PDIA6 extrapolated from the standard curve;

(PDLIM1) is the quantity of PDLIM1 extrapolated from the standard curve;

(QARS) is the quantity of QARS extrapolated from the standard curve;

(RBM4) is the quantify of RBM4 extrapolated from the standard curve; and

(WIPF1) is the quantity of WIPF1 extrapolated from the standard curve

An expression index greater than zero is diagnostic for IBD and an index less than zero is not consistent with IBD.

TABLE 10a
IBD vs normal (5-gene) Classification Matrix

| | IBD | normal |
|---|---|---|
| test > 0 | 182 | 2 |
| test ≦ 0 | 10 | 34 |

TABLE 10b
Performance Measures

| | |
|---|---|
| Accuracy | 95% |
| Sensitivity | 95% |
| Specificity | 94% |
| Positive Predictive Value | 99% |
| Negative Predictive Value | 77% |

### REFERENCES

[1] Burczynski et al. J Molec Diag 8 (1):51-61, 2006.

### EXAMPLE 3

In a subsequent study, the genes shown in Table 4 were evaluated on RBC-depleted whole blood samples obtained from an expanded set of subjects; 98 normal controls, 95 Ulcerative Colitis patients, and 97 Crohn's Disease patients. Samples were obtained from 7 clinical sites at various geographic locations within the U.S.A. The RNA expression levels of the genes were measured as described in Example 2. For each of the data subsets we evaluated the accuracy of gene combinations using logistic regression as described in Example 2.

### 3A) 6 Gene Combination

By using reverse stepwise logistic regression and only retaining genes with statistically significant associations with either IBD vs normal, or with UC vs CD, we identified a set of 6 genes (CD4, DNAJA1, MMD, PDLIM1, RBM4, WIPF1) from which a combination of 3 (CD4, PDLIM1, RBM4) is diagnostic for IBD, and from which a combination of 5 (CD4, DNAJA1, MMD, RBM4, WIPF1) is discriminative between UC and CD.

In our analyses, 190 randomly selected subjects were used to identify the significant combinations in the reverse stepwise logistic regressions, and 100 were set aside for later evaluation of the identified combinations. From the identification (or training) phase, the equation for calculating the IBD diagnostic expression index is:

Index=−0.773752+0.247525*log(CD4)−0.2753*log2(PDLIM1)−0.16045*log(RBM4)

An index greater than zero is diagnostic for IBD.

The equation for calculating the UC/CD differential diagnostic expression index is:

Index=0.45632−0.27332*log(CD4)+0.5857*log(DNAJA1)+0.23856*log(MMD)−0.0588*log(RBM4)−0.0712*log(WIPF1)

An expression index greater than zero is diagnostic for UC and an index less than zero is diagnostic for CD.

In the equations given above:

(CD4) is the quantity of CD4 extrapolated from the standard curve;

(DNAJA1) is the quantity of DNAJA1 extrapolated from the standard curve; and

(MMD) is the quantity of MMD extrapolated from the standard carve

(PDLIM1) is the quantity of PDLIM1 extrapolated from the standard curve

(RBM4) is the quantity of RBM4 extrapolated from the standard curve

(WIPF1) is the quantity of WIPF1 extrapolated from the standard curve

The performance of the IBD vs Normal index on the 100 validation subjects is summarized below:

TABLE 11
IBD vs Normal (3-gene) Classification Matrix

| | IBD | Normal |
|---|---|---|
| test > 0 | 57 | 8 |
| test ≦ 0 | 2 | 27 |

chi-squared p = 4.1 × 10⁻¹³

TABLE 12
Performance Measures

| | |
|---|---|
| Accuracy | 89.4% (80.9%-94.5%)* |
| Sensitivity | 96.6% (87.3%-99.4%) |
| Specificity | 77.1% (59.4%-89.0%) |
| Positive Predictive Value | 87.7% (76.6%-94.2%) |
| Negative Predictive Value | 93.1% (75.8%-98.9%) |

*95% confidence interval

The performance of the UC vs CD index on those IBD validation subjects subsequently identified by the above 3 gene IBD index as IBD subjects is summarized below:

TABLE 13
UC vs CD (5-gene) Classification Matrix

| | UC | CD |
|---|---|---|
| test > 0 | 24 | 4 |
| test ≦ 0 | 2 | 25 |

chi-squared p = 2.9 × 10⁻⁸

TABLE 14
Performance Measures wrt UC

| | |
|---|---|
| Accuracy | 89.1% (77.1%-94.5%)* |
| Sensitivity | 92.3% (73.4%-98.7%) |
| Specificity | 86.2% (67.4%-95.5%) |
| Positive Predictive Value | 85.7% (66.4%-95.3%) |
| Negative Predictive Value | 92.6% (74.2%-98.7%) |

*95% confidence interval

We claim

1. A biomarker consisting of between 2 and 35 different nucleic acid probe sets, including:
(a) a first probe set that selectively hybridizes under high stringency conditions to a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), WIPF1 (SEQ ID NO:10), or full complements thereof; and
(b) a second probe set that selectively hybridizes under high stringency conditions to a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), WIPF1 (SEQ ID NO:10), or full complements thereof,
wherein the first probe set and the second probe set do not selectively hybridize to the same nucleic acid target.

2. The biomarker of claim 1, including a third probe set that selectively hybridizes under high stringency conditions to a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), WIPF1 (SEQ ID NO:10), or full complements thereof,
wherein none of the first probe set, the second probe set, and the third probe set selectively hybridize to the same nucleic acid target.

3. The biomarker of claim 2, including a fourth probe set that selectively hybridizes under high stringency conditions to a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO: 7), RBM4 (SEQ ID NO:8), QARS (SEQ ID NO:9), WIPF1 (SEQ ID NO:10), or full complements thereof,
wherein none of the first probe set, the second probe set, the third probe set and the fourth probe set selectively hybridize to the same nucleic acid target.

4. A biomarker consisting of between 2 and 35 different primer pairs, including:
(a) a first primer pair capable of selectively amplifying a detectable portion of a nucleic acid target selected from the group consisting of IGH (SEQ ID NO: 11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof; and
(b) a second primer pair capable of selectively amplifying a detectable portion of a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof;
wherein the first primer pair and the second primer pair do not selectively amplify the same nucleic acid.

5. The biomarker of claim 4, including a third primer pair capable of selectively amplifying a detectable portion of a nucleic acid selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof;
wherein none of the first primer pair, the second primer pair, and the third primer pair selectively amplify the same nucleic acid target.

6. The biomarker of claim 5, including a fourth primer pair capable of selectively amplifying a detectable portion of a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8),
wherein none of the first primer pair, the second primer pair, the third primer pair, and the fourth primer pair selectively amplify the same nucleic acid target.

7. A method for diagnosing UC or CD comprising:
(a) contacting a mRNA-derived nucleic acid sample obtained from a subject suspected of having UC or CD under hybridizing conditions with 2 or more probes sets, wherein at least a first probe set and a second probe set selectively hybridize under high stringency conditions to a nucleic acid target selected from the group consisting of IGH (SEQ ID NO;11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof; wherein the first probe set and the second probe set do not selectively hybridize to the same nucleic acid target;
(b) detecting formation of hybridization complexes between the 2 or more probe sets and nucleic acid targets in the nucleic acid sample, wherein a number of such hybridization complexes provides a measure of gene expression of the nucleic acid targets; and
(c) diagnosing whether the subject is likely to have UC or CD based on the gene expression of the nucleic acid targets.

8. The method of claim 7, wherein the two or more probe sets comprise at least 3 probe sets, and wherein none of the first probe set, the second probe set, and the third probe set selectively hybridize to the same nucleic acid target.

9. The method of claim 7, wherein the two or more probe sets compose at least 4 probe sets, and wherein none of the first probe set, the second probe set, the third probe set, and the fourth probe set selectively hybridize to the same nucleic acid target

10. A method for diagnosing UC or CD comprising:
(a) contacting a mRNA-derived nucleic acid sample obtained from a subject suspected of having UC or CD under amplifying conditions with 2 or more primer pairs, wherein at least a first primer pair and a second primer pair are capable of selectively amplifying a detectable portion of a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof; wherein the first primer pair and the second primer pair do not selectively amplify the same nucleic acid target;
(b) detecting amplification products generated by amplification of nucleic acid targets in the nucleic acid sample by the two or more primer pairs, wherein the amplification products provide a measure of gene expression of the nucleic acid targets; and
(c) diagnosing whether the subject is likely to have UC or CD, based on the amplification of the nucleic acid targets.

11. The method of claim 10, wherein the two or more primer pairs comprise at least three primer pairs, wherein none of the first primer pair, the second primer pair, and the third primer pair selectively amplify the same nucleic acid target.

12. The method of claim 10, wherein the two or more primer pairs comprise at least four primer pairs, wherein none of the first primer pair, the second primer pair, the third primer pair, and the fourth primer pair selectively amplify the same nucleic acid target.

13. A method for diagnosing IBD comprising:
(a) contacting a mRNA-derived nucleic acid sample obtained from a subject suspected of having IBD under hybridizing conditions with 2 or more probes sets, wherein at least a first probe set and a second probe set selectively hybridize under high stringency conditions to a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2(SEQ ID NO:7), RBM4 (SEQ ID NO:8), QARS (SEQ ID NO:9), and WIPF1 (SEQ ID NO:10), or full complements thereof; wherein the first probe set and the second probe set do not selectively hybridize to the same nucleic acid target;
(b) detecting formation of hybridization complexes between the 2 or more probe sets and nucleic acid targets in the nucleic acid sample, wherein a number of such hybridization complexes provides a measure of gene expression of the nucleic acid targets; and
(c) diagnosing whether the subject is likely to nave IBD based on the gene expression of the nucleic acid targets.

14. The method of claim 13, wherein the two or more probe sets comprise at least 3 probe sets, and wherein none of the first probe set, the second probe set, and the third probe set selectively hybridize to the same nucleic acid target.

15. The method of claim 13, wherein the two or more probe sets comprise at least 4 probe sets, and wherein none of the first probe set, the second probe set, the third probe set, and the fourth probe set selectively hybridize to the same nucleic acid target

16. A method for diagnosing IBD comprising:
(a) contacting a mRNA-derived nucleic acid sample obtained from a subject suspected of having IBD under amplifying conditions with 2 or more primer pairs, wherein at least a first primer pair and a second primer pair are capable of selectively amplifying a detectable portion of a nucleic acid target selected from the group consisting of IGH (SEQ ID NO:11, 12, 13, 14, 15, and/or 16), MMD (SEQ ED NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof; wherein the first primer pair and the second primer pair do not selectively amplify the same nucleic acid target;
(b) detecting amplification products generated by amplification of nucleic acid targets in the nucleic acid sample by the two or more primer pairs, wherein the amplification products provide a measure of gene expression of the nucleic acid targets; and
(c) diagnosing whether the subject is likely to have IBD based on the amplification of the nucleic acid targets.

17. The method of claim 16, wherein the two or more primer pairs comprise at least three primer pairs, wherein none of the first primer pair, the second primer pair, and the third primer pair selectively amplify the same nucleic acid target.

18. The method of claim 16, wherein the two or more primer pairs comprise at least four primer pairs, wherein none of the first primer pair, the second primer pair, the third primer pair, and the fourth primer pair selectively amplify the same nucleic acid target.

19. A method for diagnosing IBD and providing a differential diagnosis of UC or CD comprising:
(a) contacting a mRNA-derived nucleic acid sample obtained from a subject suspected of having IBD under hybridizing conditions with 2 or more probes sets, wherein at least a first probe set and a second probe set selectively hybridize under high stringency conditions to a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof; wherein the first probe set and the second probe set do not selectively hybridize to the same nucleic acid target;
(b) detecting formation of hybridization complexes between the 2 or more probe sets and nucleic acid targets in the nucleic acid sample, wherein a number of such hybridization complexes provides a measure of gene expression of the nucleic acid targets;
(c) diagnosing whether the subject is likely to have IBD based on the gene expression of the nucleic acid targets; and
(d) further diagnosing whether the IBD patient has UC or CD based on the gene expression of the nucleic acid targets.

20. A method for diagnosing IBD and providing a differential diagnosis of UC or CD comprising:
(a) contacting a mRNA-derived nucleic acid sample obtained from a subject suspected of having IBD under amplifying conditions with 2 or more primer pairs, wherein at least a first primer pair and a second primer pair are capable of selectively amplifying a detectable portion of a nucleic acid target selected from the group consisting of IGH (SEQ ID NO:11, 12, 13, 14, 15, and/or 16), MMD (SEQ ID NO:2), PDLIM1 (SEQ ID NO:3), PDIA6 (SEQ ID NO:4), CD4 (SEQ ID NO:5), DNAJA1 (SEQ ID NO:6), HBA2 (SEQ ID NO:7), RBM4 (SEQ ID NO:8), QARS (SEQ ID NO:9), and WIPF1 (SEQ ID NO:10), or full complements thereof; wherein the first primer pair and the second primer pair do not selectively amplify the same nucleic acid target;
(b) detecting amplification products generated by amplification of nucleic acid targets in the nucleic acid sample by the two or more primer pairs, wherein the amplification products provide a measure of gene expression of the nucleic acid targets; and
(e) diagnosing whether the subject is likely to have IBD based on the amplification of the nucleic acid targets; and
(d) further diagnosing whether the IBD patient has UC or CD based on the amplification of the nucleic acid targets.


SEQUENCE LISTING (16 sequences)

SEQ ID NO: 1 - skipped

SEQ ID NO: 2 - DNA, 2723 bp, Homo sapiens
gggcggggcgcaggagccccggggcggaggagccggggaggcgggaggcgggaggcgggaggtgttggggccgttgaagcggcctccctcccgcccccagccgcccggtctggccccagccctgtcccgacccccggcctggcccactccgaccctacccggccgaagggttccgctggacacgcaggcggcctccggagcagcccaagcccatgagggccgcgcgcccggccgccggtgctgacgagacggagctcctggcccccgaggaggagcagaggatcaatgcggttcaagaatcgattccagcggttcatgaaccatcgagctccagccaatggccgctacaagccaacttgctatgaacatgctgctaactgttacacacacgcattcctcattgttccggccatcgtgggcagtgccctcctccatcggctgtctgatgactgctgggaaaagataacagcatggatttatggaatgggactctgtgccctcttcatcgtttctacagtatttcacattgtatcatggaaaaagagccacttaaggacagtggagcattgttttcacatgtgtgatagaatggttatctatttcttcattgctgcttcttatgctccatggttaaatcttcgtgaacttggacccctggcatctcatatgcgttggtttatctggctcatggcagctggaggaaccatttatgtatttctctaccatgaaaaatataaggtggttgaactctttttctatctcacaatgggattctctccagccttggtggtgacatcaatgaacaacaccgatggacttcaggaacttgcctgtgggggcttaatttattgcttgggagttgtgttcttcaagagtgatggcatcattccatttgcccacgccatctggcacctgtttgtggccacggcagctgcagtgcattactacgccatttggaaatacctttaccgaagtcctacggactttatgcggcatttatgaccaatctgtactaattctccaaaccagtattatttcaattatggcacttgggagtggggtgagagctaaacattgcacagggaaagaaaaaaaataactgcactgactttatatcttttgaatataattactgtgaaagtataaaggctgtgttctggaattttctgcctcacagcaaataaataaggtagtgaattaattattcattccattccactatcatgaaggactctgaatagacttggccaactgatgtttacaaaccagacttttatattttaattttacagattttactacatgatttttctaaattactatgtcaggttgtaaaagtcagtgcaataacaaaccttcctttttaagaagaaaattgtttctattactttcccattcactaggtaaagaatcatggacagaacttacactactttttaccatgtttcatcttggcataacatggttcttttttaaatagaaactttagttttttgtaaatttttaaaaaaatatttcattgatatgcatctctgcaggtcctcattcatgttgtaaatttttgcagcaagcagtcaacattccacaaacgaacaaacattatacctcttctgatagttttattaagcatggagaaattgccaatttttaaaaactgcagttttccaaacttttctgccaacctcttactctgaattcagtgctgctttgggacatatacttgacctagcttggtttaccagtgatggaaaagtattttgatatcattaactttttcaaaagatccaactttttctctatgcctttgccacattctcttcagggtctctttccacagtggataaatgttttttctgtattatgacagtattgttgtgatggccatctgctggaaactcctgaagagcattatgtattacagtgagcagttgtattgcctgtttggtgcccaatggttaagtcattgtcacttagctttatattgtcagtttgatatttattttaaattgtggaactagatgcataaattcacatttctgcctttcctttgcatcttctcatatattgtgtttttttttttttttcctagaaaaaatatttaaagcattgtttgacaggtagaaactcatgtatctgtagtccatgagttatatcctggctcagtggagtgatatttatgtattatttttacttttctctcagtgtcttatattaagattaacatgttgttaatagttgctttgttgattaatctctcttgttggtgttttaataaatgaaataggcttgcctttagatcgggtgctgatattgcctgtttcctagtaatgggctgatcaaatgatcagtggaattcttggtttgatgataaccttattaattgaaattttttactgatgtggctttaaaagaggtttattttgtatatgtttagaactctctgattttgatgaattatatgggaatgagaaacagaagaagtggtatttgctggcgagttaaataggcaaggtacccagtgataacaccaaccaaaccactcctatctgcatgattctgaacatctggatgcctgttgttttactgtgtatattttatttttaatatattaactttgtggattcatttaaggtctactcaaaagtaacactgtcaaaaccactaatatgtatgtaaaaattgtgctgtatactacaataaagttgttacttggatttgttcca

SEQ ID NO: 3 - DNA, 1462 bp, Homo sapiens
gcccgcgccgcgccgctctttctccgacagctgccgggggtgccctgcaagctgttccgcgcgtcctgcccgtctgtccccgcgggtcgtcgcccgccacagccgcgccatgaccacccagcagatagacctccagggcccggggccgtggggcttccgcctcgtgggcggcaaggacttcgagcagcctctcgccatttcccgggtcactcctggaagcaaggcggctctagctaatttatgtattggagatgtaatcacagccattgatggggaaaatactagcaatatgacacacttggaagctcagaacagaatcaaaggctgcacagacaacttgactctcactgtagccagatctgaacataaagtctggtctcctctggtgacggaggaagggaagcgtcatccatacaagatgaatttagcctctgaaccccaggaggtcctgcacataggaagcgcccacaaccgaagtgccatgccctttaccgcctcgcctgcctccagcactactgccagggtcatcacaaaccagtacaacaacccagctggcctctactcttctgaaaatatctccaacttcaacaatgccctggagtcaaagactgctgccagcggggtggaggcgaacagcagacccttagaccatgctcagcctccaagcagccttgtcatcgacaaagaatctgaagtttacaagatgcttcaggagaaacaggagttgaatgagcccccgaaacagtccacgtctttcttggttttgcaggaaatcctggagtctgaagaaaaaggggatcccaacaagccctcaggattcagaagtgttaaagctcctgtcactaaagtggctgcgtcgattggaaatgctcagaagttgcctatgtgtgacaaatgtggcactgggattgttggtgtgtttgtgaagctgcgggaccgtcaccgccaccctgagtgttatgtgtgcactgactgtggcaccaacctgaaacagaagggccatttctttgtggaggatcaaatctactgtgagaagcatgcccgggagcgagtcacaccacctgagggttatgaagtggtcactgtgttccccaagtgagccagcagatctgaccactgttctccagcaggcctctgctgcagctttttctctcagtgttctggccctctcctctcttgaaagttctctgcttactttggttttccctctgcttgtaaaacattgagtcccctccctgccttggttaattgactcacaccagctgtgcgatgcccgcttttacaattaaaggaaaactgttttgttcagtgtcaccttgtcagcaacactgtgtcccttcgccccaccgttcttctctgctgcatttggacatcagccaaatttgaacccaatcaaatataacgtgtctgacactgattttgtttttactcaataaatgtatagactacaaaaaaaaaaaaa

SEQ ID NO: 4 - DNA, 2344 bp, Homo sapiens
ggcccgcccccgcgcggccgcccctcggaccaccggactggcctggggcgggacgtgggcgcgggggcgcggcgtgcggcacgctgcagggctgaagcggcggcggcggtggggactgcacgtagcccggcgctcggcatggctctcctggtgctcggtctggtgagctgtaccttctttctggcagtgaatggtctgtattcctctagtgatgatgtgatcgaattaactccatcgaatttcaaccgagaagttattcagagtgatagtttgtggcttgtagaattctatgctccatggtgtggtcactgtcaaagattaacaccagaatggaagaaagcagcaactgcattaaaagatgttgtcaaagttggtgcagttgatgcagataagcatcattccctaggaggtcagtatggtgttcagggatttcctaccattaagatttttggatccaacaaaaacagaccagaagattaccaaggtggcagaactggtgaagccattgtagatgctgcgctgagtgctctgcgccagctcgtgaaggatcgcctcgggggacggagcggaggatacagttctggaaaacaaggcagaagtgatagttcaagtaagaaggatgtgattgagctgacagacgacagctttgataagaatgttctggacagtgaagatgtttggatggttgagttctatgctccttggtgtggacactgcaaaaacctagagccagagtgggctgccgcagcttcagaagtaaaagagcagacgaaaggaaaagtgaaactggcagctgtggatgctacagtcaatcaggttctggcctcccgatacgggattagaggatttcctacaatcaagatatttcagaaaggcgagtctcctgtggattatgacggtgggcggacaagatccgacatcgtgtcccgggcccttgatttgttttctgataacgccccacctcctgagctgcttgagattatcaacgaggacattgccaagaggacgtgtgaggagcaccagctctgtgttgtggctgtgctgccccatatccttgatactggagctgcaggcagaaattcttatctggaagttcttctgaagttggcagacaaatacaaaaagaaaatgtgggggtggctgtggacagaagctggagcccagtctgaacttgagaccgcgttggggattggagggtttgggtaccccgccatggccgccatcaatgcacgcaagatgaaatttgctctgctaaaaggctccttcagtgagcaaggcatcaacgagtttctcagggagctctcttttgggcgtggctccacggcacctgtaggaggcggggctttccctaccatcgttgagagagagccttgggacggcagggatggcgagcttcccgtggaggatgacattgacctcagtgatgtggagcttgatgacttagggaaagatgagttgtgagagccacaacagaggcttcagaccattttcttttcttgggagccagtggatttttccagcagtgaagggacattctctacactcagatgactctaccagtggccttttaaccaagaagtagtacttgattggtcatttgaaaacactgcaacagtgaacttttgcatctcaagaaaacattgaaaaattctatgaattgttgtagccggtgaattgagtcgtattctgtcacataatattttgaagaaaacttggctgtcgaaacatttttctctctgactgctgcttgaatgttcttggaggctgtttcttatgtatgggttttttttaatgtgatcccttcatttgaatattaatggctttttccattaaagaataaaatattttggacaatgccgataaatgtatgaagttagtatccacatcataaattcagagtgatgtttagcagtaaatcaatattttgaagtgatacacagatgtctttcctccccacaaacttttttaaacaaaaaacaagacctcttttctttagatggtgccacctatgcccaccacaacagagattttacatggaaaccgggctcagtgagaactgatttcctgcccaatatttgtctttgggctgtctctagtgactaattattaaggaatctagctggttatacagttcaaggctttctatgttgttaatgaacctcaaaatagccgttaagacatgaaatacagcagcaggttaccaatgcgaacaggtagttcgcatttatgtaaaacattcagaaaatgaagttttgaatttgttggaacattcaaaggacttgagagcattttattgtaacttaaaaaaataaatacaactgtcactaaaaaaaaaaaaaaaaaa

SEQ ID NO: 5 - DNA, 3103 bp, Homo sapiens
ccaggcctagggtgtggaggagccttgccatcgggcttcctgtctctcttcatttaagcacgactctgcagaaggaacaaagcaccctccccactgggctcctggttgcagagctccaagtcctcacacagatacgcctgtttgagaagcagcgggcaagaaagacgcaagcccagaggccctgccatttctgtgggctcaggtccctactggctcaggcccctgcctccctcggcaaggccacaatgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgtcctcaccggtttcagaagacatgtagccccatttgaggcacgaggccaggcagatcccacttgcagcctccccaggtgtctgccccgcgtttcctgcctgcggaccagatgaatgtagcagatccccagcctctggcctcctgttcgcctcctctacaatttgccattgtttctcctgggttaggccccggcttcactggttgagtgttgctctctagtttccagaggcttaatcacaccgtcctccacgccatttccttttccttcaagcctagcccttctctcattatttctctctgaccctctccccactgctcatttggatcccaggggagtgttcagggccagccctggctggcatggagggtgaggctgggtgtctggaagcatggagcatgggactgttcttttacaagacaggaccctgggaccacagagggcaggaacttgcacaaaatcacacagccaagccagtcaaggatggatgcagatccagaggtttctggcagccagtacctcctgccccatgctgcccgcttctcaccctatgtgggtgggaccacagactcacatcctgaccttgcacaaacagcccctctggacacagccccatgtacacggcctcaagggatgtctcacatcctctgtctatttgagacttagaaaaatcctacaaggctggcagtgacagaactaagatgatcatctccagtttatagaccagaaccagagctcagagaggctagatgattgattaccaagtgccggactagcaagtgctggagtcgggactaacccaggtcccttgtcccaagttccactgctgcctcttgaatgcagggacaaatgccacacggctctcaccagtggctagtggtgggtactcaatgtgtacttttgggttcacagaagcacagcacccatgggaagggtccatctcagagaatttacgagcagggatgaaggcctccctgtctaaaatccctccttcatcccccgctggtggcagaatctgttaccagaggacaaagcctttggctcttctaatcagagcgcaagctgggagcacaggcactgcaggagagaatgcccagtgaccagtcactgaccctgtgcagaacctcctggaagcgagctttgctgggagagggggtagctagcctgagagggaaccctctaagggacctcaaaggtgattgtgccaggctctgcgcctgccccacaccctcccttaccctcctccagaccattcaggacacagggaaatcagggttacaaatcttcttgatccacttctctcaggatcccctctcttcctacccttcctcaccacttccctcagtcccaactccttttccctatttccttctcctcctgtctttaaagcctgcctcttccaggaagacccccctattgctgctggggctccccatttgcttactttgcatttgtgcccactctccacccctgctcccctgagctgaaataaaaatacaataaacttactataaagatgc

SEQ ID NO: 6 - DNA, 1538 bp, Homo sapiens
ggacttccggcgggtgacgcgcccgggttcggctacaaaagaggacggctgcggcgcgccgggcggaactttccagaacgctcggtgagaggcggaggagcggtaactaccccggctgcgcacagctcggcgctccttcccgctccctcacacaccggcctcagcccgcaccggcagtagaagatggtgaaagaaacaacttactacgatgttttgggggtcaaacccaatgctactcaggaagaattgaaaaaggcttataggaaactggctttgaagtaccatcctgataagaacccaaatgaaggagagaagtttaaacagatttctcaagcttacgaagttctctctgatgcaaagaaaagggaattatatgacaaaggaggagaacaggcaattaaagagggtggagcaggtggcggttttggctcccccatggacatctttgatatgttttttggaggaggaggaaggatgcagagagaaaggagaggtaaaaatgttgtacatcagctctcagtaaccctagaagacttatataatggtgcaacaagaaaactggctctgcaaaagaatgtgatttgtgacaaatgtgaaggtagaggaggtaagaaaggagcagtagagtgctgtcccaattgccgaggtactggaatgcaaataagaattcatcagataggacctggaatggttcagcaaattcagtctgtgtgcatggagtgccagggccatggggagcggatcagtcctaaagatagatgtaaaagctgcaacggaaggaagatagttcgagagaagaaaattttagaagttcatattgacaaaggcatgaaagatggccagaagataacattccatggtgaaggagaccaagaaccaggactggagccaggcgatattatcattgtgttagatcagaaggaccatgctgtttttactcgacgaggagaagaccttttcatgtgtatggacatacagctcgttgaagcactgtgtggcttccagaagccaatatctactcttgacaaccgaaccatcgtcatcacctctcatccaggtcagattgtcaagcatggagatatcaagtgtgtactaaatgaaggcatgccaatttatcgtagaccatatgaaaagggtcgcctaatcatcgaatttaaggtaaactttcctgagaatggctttctctctcctgataaactgtctttgctggaaaaactcctacccgagaggaaggaagtggaagagactgatgagatggaccaagtagaactggtggactttgatccaaatcaggaaagacggcgccactacaatggagaagcatatgaggatgatgaacatcatcccagaggtggtgttcagtgtcagacctcttaatgggccagtgaataacactcactgctggcatttaatgtgcagtagtgaatgagtgaaggactgtaatcataatatgctcactacttgctcttgtttttgttttaataaactatagtagtgttttaaaaagttaaatgaagaataaacgcaaatataaaagc

SEQ ID NO: 7 - DNA, 622 bp, Homo sapiens
cataaaccctggcgcgctcgcgggccggcactcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcagcaaaaaaaaaaaaaaaaaa

SEQ ID NO: 8 - DNA, 1678 bp, Homo sapiens
agagcactgctgcggccgccgccattttagcgttttgtcagaagcgtccgcgccgcgaggaggaggccctgctggtttctgtgcgggctcttgtcaggatggtgaagctgttcatcggaaacctgccccgggaggctacagagcaggagattcgctcactcttcgagcagtatgggaaggtgctggaatgtgacatcattaagaattacggctttgtgcacatagaagacaagacggcagctgaggatgccatacgcaacctgcaccattacaagcttcatggggtgaacatcaacgtggaagccagcaagaataagagcaaaacctcaacaaagttgcatgtgggcaacatcagtcccacctgcaccaataaggagcttcgagccaagtttgaggagtatggtccggtcatcgaatgtgacatcgtgaaagattatgccttcgtacacatggagcgggcagaggatgcagtggaggccatcaggggccttgataacacagagtttcaaggcaaacgaatgcacgtgcagttgtccaccagccggcttaggactgcgcccgggatgggagaccagagcggctgctatcggtgcgggaaagaggggcactggtccaaagagtgtccgatagatcgttcaggccgcgtggcagacttgaccgagcaatataatgagcaatacggagcagtgcgtacgccttacaccatgagctatggggattcattgtattacaacaacgcgtacggagcgctcgatgcctactacaagcgctgccgtgctgcccggtcctatgaggcagtggcagctgcagctgcctccgtgtataattacgcagagcagaccctgtcccagctgccacaagtccagaatacagccatggccagtcacctcacctccacctctctcgatccctacgatagacacctgttgccgacctcaggagctgctgccacagctgctgctgcagcagcagccgctgctgctgttactgcagcttccacttcatattacgggcgggatcggagccccctgcgtcgcgctacagccccagtccccactgttggagagggctacggttacgggcatgagagtgagttgtcccaagcttcagcagccgcgcggaattctctgtacgacatggcccggtatgagcgggagcagtatgccgatcgggcgcggtactcagccttttaaagcttgaggtgggatgtgtgtgggctgaaattccgagctgcggttgtgcatgagaatacacccttcgtggtaccccatctccgggacgttctcggctctgtgcgttcagtccctcaggaaccgtggaccttaatttaccttgctaagttcagaccttctcttcctttcctttcctttcctctcctgcccattttcctgttcttctgtccttcaatacttctgtagcttcccattcatgttctcttctcccagcaggcctcattgtgtgcagaaactgtggtgggggctgtgctgtctcctccctgcctcctgcctcctgcggctgttggatttgggaatgaccttggtgagagtctcactgctccagggtctctttttggtccaaaggctagacctatagagttggatcactttttttctttccggtgaaataaatggtttttcaacttagaaaaaaaaaaaaaaaaaaaaaaaaaaaaaaaaaa

SEQ ID NO: 9 - DNA, 2437 bp, Homo sapiens
ctgcaatggcggctctagactccctgtcgctcttcactagcctcggcctgagcgagcagaaggcccgcgagacgctcaagaactcggctctgagcgcgcagctgcgcgaggccgctactcaggctcagcagaccctgggttccaccattgacaaagctaccgggatcctgttatatggcttggcctcccgactcagggatacccggcgtctctccttccttgtaagctacatagccagtaagaagatccacactgagccccagctaagcgctgcccttgagtatgtgcggagtcaccccttggaccccatcgacactgtggacttcgagcgggaatgtggcgtgggtgtcattgtgaccccagagcagattgaggaggctgtggaggctgctattaacaggcaccggccccagctcctggtggaacgttaccatttcaacatggggctgctgatgggagaggctcgggctgtgctgaagtgggcagatggcaaaatgatcaagaatgaagtggacatgcaggtcctccaccttctgggccccaagttggaggctgatctggagaagaagttcaaggtggcaaaagctcggctagaagaaacagaccggaggacggcaaaggatgtggtggagaatggcgagactgctgaccagaccctgtctctgatggagcagctccggggggaggcccttaagttccacaagcctggtgagaactacaagaccccaggctatgtggtcactccacacaccatgaatctactaaagcagcacctggagattactggtgggcaggtacgtacccggttcccgccagaacccaatggaatcctgcatattggacatgccaaagccatcaatttcaactttggctatgccaaggccaacaatggcatctgttttctgcgttttgatgacaccaaccctgagaaggaggaagcaaagttcttcacggccatctgtgacatggtagcctggctaggctacacaccttacaaagtcacatatgcgtctgactattttgaccagctatatgcgtgggctgtggagctcatccgcaggggtctggcttatgtgtgccaccagcgaggagaggagctcaaaggccataatactctgccttcaccctggagagaccgtcccatggaggagtcactgctgctctttgaggcaatgcgcaagggcaagttttcagagggcgaggccacactacggatgaagctggtgatggaggatggcaagatggaccctgtagcctatcgagtcaagtatacaccacaccaccgcacaggggacaaatggtgcatctatcccacctacgactacacacactgcctctgtgactccatcgagcacatcactcactcactctgcaccaaggaattccaggcccgacgctcttcctacttctggctttgcaatgcactggacgtctattgccctgtgcagtgggagtatggccgcctcaacctgcactatgctgttgtctctaagaggaagatcctccagcttgtagcaactggtgctgtgcgggactgggatgacccacggctctttacactcacggccctgcgacggcggggcttcccacctgaggccatcaacaacttctgtgcccgggtgggagtgactgtggcacaaaccacaatggagccacatcttctagaagcctgtgtgcgtgatgtgctgaatgacacagccccacgagccatggctgtgctggagtcactacgggtcatcatcaccaactttcctgctgccaagtccttggacatccaggtgcccaacttcccagctgatgagaccaaaggcttccatcaggttccctttgcacccattgtcttcattgagaggactgacttcaaggaggagccagagccaggatttaagcgcctggcttggggccagcctgtgggcctgaggcatacaggctacgtcattgagctgcagcatgttgtcaagggccccagtggttgtgtagagagtctggaggtgacctgcagacgggcagatgctggagagaagccaaaggcctttattcactgggtgtcacagcctttgatgtgtgaggttcgcctctatgagcgactattccagcacaagaaccctgaagatcctactgaggtgcctggtggatttttaagtgacctgaacctggcatcactacacgtggtggatgcagcattagtggactgctctgtggccctggcaaaacccttcgacaagttccagtttgagcgtcttggatatttctccgtggatccagacagccatcagggaaagcttgtctttaaccgaactgtcacactgaaggaagacccaggaaaggtgtgagctggaagcactgaacctacctcatcctcctggagggtgtggctaccctcgccaccccaaattccatgtcaataaagaacagctaaattctcctagaaaaaaaa

SEQ ID NO: 10 - DNA, 4664 bp, Homo sapiens
gcagcctcccggcgctgagcgcttttcctgcccgcccggctcagccctgcggaccccgggagaagtttcccagaaaaaatgcccagcgcggcgcggggctgcggagtcgtccggagccgctgcgcgatttatcagcaagactgttgaacgcataactgcccaagatgcctgtccctccccctccagcacccccgccgcccccgacgtttgcactggccaatacagagaagcctaccttgaataagacagagcaggctgggagaaatgctctcctttctgatatcagcaaagggaagaaactaaagaagacggtcaccaatgacagaagtgcaccaatactggacaaacctaaaggagctggtgctggaggcggtggtggtggctttggtggaggcggcggatttggcggaggaggtggtggcggaggcggtggaagttttggagggggcggacctccaggtctgggaggattgttccaggctggaatgccgaagctgagatccacggccaacagggataatgattctggaggaagccgaccaccattgttgccaccgggaggaagatccacatctgcgaaacccttttcacccccaagtggcccagggaggtttcctgtgccttctccaggccacagaagtggtcccccagagcctcagaggaaccgaatgccgcccccaaggcccgacgtgggctcaaagcctgatagcattcctcctccagtacctagtactccaagacccattcaatcaagtccgcacaaccgggggtccccaccagtgcccggaggccccaggcagcccagccccgggcccactcctccccctttccctggaaaccgcggcactgctttgggaggaggctcaatacgtcagtcccccttgagctcctcctcgcccttctccaaccggcctcccctgccgcctacccccagcagggccttggatgacaaaccccctccaccacctcctccagtgggcaacaggccctccatccacagggaagcggttccccctcctcctcctcagaacaacaagcctccagtgccttccactccgcggccttcggcctcctcacaggccccacctccgccgccacctcccagcaggcccgggccgcctcctctgcctccaagttccagcggcaatgacgaaaccccaagactcccacagcggaatctgtccctcagttcgtccacgcccccgttaccttcgccaggacgttcaggtcctcttcctcccccgcccagtgagagacccccacctccagtgagggacccgccaggccgatcaggccccctcccaccacctcctccagtaagcagaaacggcagcacatctcgggccctgcctgctacccctcagttgccatccaggagtggagtagacagtcccaggagtggacccaggcctccccttcctcctgataggcccagtgctggggcacctcccccacctccaccatcaacatctattagaaatggcttccaagactctccatgtgaagatgagtgggaaagcagattctacttccatccgatttccgatttgccacctccagagccatatgtacaaacgaccaaaagttatcccagcaaactggcaagaaacgaaagccggagtggatccaaccgaagagaaaggggtgctccaccactccctcccatcccgaggtgatctttgcctgctcttctctacccaagctcaagagctgcttctgttgctatctaagaactgcataccctcctccctgcttcttcccttgtgcctcatgtatgggcaggaggaaaggtgggagggggagtgggaatatgcgtgtgtgggtgggaatcggtaagaaatgcacctagcttttcatattgtgtttattctccaggctattgcttgcttcagctgcagcctgcctgtgctggctgctggggtcgataggcttttgtcgtaataggcagagatgacttgcatcccagctttccaccaaccaaattcaaacattcactgcttatttgttacagactgtaattattaaagtccctgagagctgttttctcccgttcctttttcgcatgcttggcctcctctctgtttctatgaaccacagaccacctaagcaagctgctgagtaagggctcactggaaacttgcagtcacaggatgtccaatctttggcagtccgagcttggctctaggacagagctgtccaatagaaatataatgtgagccccatatacaatttttacatttctaatatattttaaacaagtgaagttaatatgcatccaaaatatttcaacctgtaatcaacataaaattttaatgagatattttatattattttttggtactgaatcttcaaaatccagagtgtattttacacttaccgcacatctccattcagactagtcacatttttaagtgctcagtagccacatgtggctggtggctactggattagacagcacgagtctggaagatggaagctagtgcagaaacctcttgtttaaaaaacaaaaaaggcaagatgggcttgagcgattcaagaggcaactaaaaataaaattaggacccagcaccttgtttgacacacagtttgaccttcgattttcctcccttaacttccctcttcccttaatatctgtatacaagtgttgcttcaaagtaccaaggtcagaaattgattcagtacggtttactaaagtcatgtggaataaagccattggaaacaaatggaaagcctgtcgggacttctgggctcagaaccagctggctcacgcactccacttgtcagctggacttctgccttgtgaaatggaagcagcctttgttcctttctggctgagcaagctcctgaggctgggagagactaggaaggcttggtaggaggggaaaaaagtcaggaaaagatatcaaatcagaaacatggaagaagaagggaaccgatttgagttggtgggcaaaactctaaaaatctaaatctgatgcttatgtaagggttgagcgaattagggagattgctagtggaaattggagggaatttgttttgcatcatttgtctaggatctatgcaaatatagctccactaaaggaccatagggaagagccagccttgccttttcttatatgattttgtttacaaaattttactgggacttttaaatctagctatagagttgggaaaaaatatttccacttagatattttacatggttttgtttaaaattaccattacttgttttttaaaaacacatgaccacatatgtatatgtatatctacctaaacattgtatcatggtttcagtatgttattcatgtattactgggagatgctaccaagaaaccaacccaaagaaaattctgaaaaatacatttctatttatagaataaatgtttcatttatataaaagcaaaagaacttagagttctaataaatgggatgtctaataaattatgaagttactgatttgaatatattatatttttataacttccttgccaaagtcctgatttagtacattagagaacctgtgtttcctctctcctctaccattcatctctcttccatacagtcatttgggctttttactcaaagagaatcaagaaataataaggtataacaagcttggcaaagtgttggctttttaaaaaaaaatttttttaatctctagcagtttggtaatttagcagcatcatttatttgggattcttttatctgatttcaacagtgaaaaacatccctatgataaagcctaatgacccatttcacaaaagatggaatttgcccttcctagaaaatatgacggagaaaagtctgactcagagaaagtgagtctgaattttataaggggtagtaagaattggacaattcctttgcatatctgaacttggcaggtaccgttctaaatctgaaacagggtgatagctcaaagttgccattcatccagaatagattgttttagaatgtagtgtttaagtgactgtttcattaatacacctacaccctttctttgaaagtttgcaacctaattgcatctaaaactatgaataagttctgtggtaaaatcttaaactatggaaaattacaaaaatgaatttttcttccctgaaatcagagcttacatgtgtgtttttttataacattttcagataaatgtattcaacatgtaatacagtattttaacattcacctcttattttatattgaaatgtattacagtattaaaactcagtgttcagtatttatttcactatgcattttatttagtaaaagccaggagaaatgtttaatccaatggtgccttactttgtgatttaaaagaaatcaacttttttttatgtctaagtagtagattatttgcatatttgtaaaaactgttaggtctttatattttaaagtgtaataccagttttgttattttagtagcagaaatgggatgattgttaaagttccccaaaaatgttggcatgaaattaatttttccctccttatagtcaaggaccgtagaggaagaaaaacttttttttcataccatgcactatgtaaacagacacattttgctatctgtgtcatcaggatagtgtaagtggtagggtagagactaccctagacatctgcatctttgtaagttagccagacaataaagaaaagcagaatgaaaaaaaaaaaaaaaa

SEQ ID NO: 11 - DNA, 1599 bp, Homo sapiens
tctaaagaagcccctgggagcacagctcatcaccatggactggacctggaggttcctctttgtggtggcagcagctacaggtgtccagtcccagatgcaggtggtgcagtctggggctgaagtaaagaagcctgggtcctcggtgacggtctcctgcaaggcatctggaggcaccttcagcaactatgctatcagctgggtgcgacaggcccctggacaagggcttgagtggatgggagggatcatccctctttttggtacaccaacctactcacagaacttccagggcagagtcacgattaccgcggacaaatccaccagcacagcccacatggagctgatcagcctgagatctgaggacacggccgtgtattactgtgcgacagatcgctacaggcaggcaaattttgaccgggcccgggttggctggttcgacccctggggccagggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccgtgtacatacttcccgggcgcccagcatggaaataaagcacccagcgctgccctgggcccctgcg

SEQ ID NO: 12 - DNA, 1604 bp, Homo sapiens
caccatggactggacctggaggttcctctttgtggtggcagcagttacaggtgtccagtcccaggtccagctggtgcagtctggggctgaggtgaagaagcctgggtcctcagtgaaggtctcctgcaaggcctctggagacaccttcactaactatgctatcagctgggtacgacaggcccctggacaaggacttgagtggatgggggggatcatccctatctttggaacaacagactacgcacagaatttccaggacagagtcacaattaccgcggacgaatctacgaatttattctctctggaaataaataacctgagatctaaagacacggccatgtattattgtgtgagagagtccttcactacggtttttggagtgccgaccctccattaccttgactcctggggccagggaacgctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccaggctctcggggtcgcgcgaggatgcttggcacgtaccccgtgtacatacttcccgggcgcccagcatggaaataaagcacccagcgctgccctgggaaaaaaaaaaaaaaaaaaaaaaaaaaaaaaaaaaaaaaaaaaaaaa

SEQ ID NO: 13 - DNA, 1656 bp, Homo sapiens
ggggacataacaaccacattcctcctctaaagaagcccctgggagcacagctcatcaccatggactggacctggaggttcctctttgtggtggcagcagctacaggtgtccagtcccaggtgcaactggtgcagtctggggcggaggtgaagaagcctgggtcctcggtgaaagtctcctgcaaggcctctggaggctcccccggcagttttgtcattagttgggtgcgacaggcccctggacaagggctcgcgtggatgggggggatcatcccagcctttgacataacaaagtacgcacagaattttcaggacagagtcaccatttctgcggacgaatctacggacacagcctacatggagctgcgaagcctgagatctgaggacacggccacatattactgtgcgagagatctcgcactttacgagctttggagtggcttccacacggacgagaagtactacggcttggacgtctggggccaagggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgcgacggccggcaagcccccgctccccgggctctcgcggtcgcacgaggatgcttggcacgtaccccgtctacatacttcccaggcacccagcatggaaataaagcacccaccactgccctgaaaaaaaaaaaaaaaaaaaaaaaaaaaaaa

SEQ ID NO: 14 - DNA, 1581 bp, Homo sapiens
tggacctcctgcacaagaacatgaaacacctgtggttcttcctcctgctggtggcagctcccagatgggtcctgtcccaggtgcagctgcaggagtcgggcccaggactggtgaagccttcggagaccctgtccctcacctgcactgtctctggaggctccatcagtggttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagaggtcggttcacctactttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcatggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgaaaaaaaaaaaaaaaaaaaaaaaaaaaaa

SEQ ID NO: 15 - DNA, 1589 bp, Homo sapiens
cctgtgcaagaacatgaaacacctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtgattactactggagctggatccgccagcccccagggaagggcctggagtggattgggtacatctattacagtgggagcacctactacaacccgtccctcaagagtcgagttaccatatcactagacacgtccaagaaccagttctccctgaagatgaactctgtgactgccgcagacacggccgtgtatttctgtgccagagcgggcgtttgggggagttttcgttcctgggcgattgatggttttaatatctggggccaagggacaatggtcaccgtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaagcacccagcgcttccctgggaaaaaaaaaaaaaaaaaaa

SEQ ID NO: 16 - DNA, 1593 bp, Homo sapiens
gggggctttctgagagtcatggacctcctgtgcaagaacatgaagcacctgtggttcttcctcctgctggtggcggctcccagatgggtcctgtcccggctgcagctgcaggagtcgggcccaggcctgctgaagccttcggtgaccctgtccctcacctgcactgtctcgggtgactccgtcgccagtagttcttattactggggctgggtccgtcagcccccagggaagggactcgagtggatagggactatcaattttagtggcaatatgtattatagtccgtccctcaggagtcgagtgaccatgtcggcagacatgtccgagaactccttctatctgaaattggactctgtgaccgcagcagacacggccgtctattattgtgcggcaggacacctcgttatgggatttggggcccactggggacagggaaaactggtctccgtctctccagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgcaaaaaaaaaaaaaaaaaaaaaaaaaaaa